(19) United States
(12) Patent Application Publication
(10) Pub. No.: US 2010/0293671 A1
(43) Pub. Date: Nov. 18, 2010

(54) Composition and Method for Prolonging the Shelf Life of Banana by Using Interfering RNA

(75) Inventors: Pung-Ling Huang, Taipei City (TW); Yi-Yin Do, Taipei City (TW); Yu-Chen Liao, Taipei County (TW); Yi-Yu Lin, Taipei County (TW); Shan-Shin Lee, Taipei County (TW); Wei-Fen Huang, Taipei County (TW)

Correspondence Address: SCHMEISER OLSEN & WATTS, 18 E UNIVERSITY DRIVE, SUITE # 101, MESA, AZ, 85201, US

(73) Assignee: NATIONAL TAIWAN UNIVERSITY, Taipei City (TW)

(21) Appl. No.: 12/465,496

(22) Filed: May 13, 2009

(51) Int. Cl. C12N 15/82; C12N 15/00; C12N 5/04; A23L 3/34

(52) U.S. Cl. 800/292; 435/320.1; 435/419; 800/294; 800/293; 426/321

(57) ABSTRACT

The invention relates to a composition and method for prolonging the shelf life of banana by using interfering RNA. Said method transfers a control cassette for Musa spp. ACC oxidase into banana by a novel gene transfer method, wherein said composition comprises an interfering RNA, a gene transfer expression vector and pharmaceutically acceptable carrier. Said interfering RNA can inhibit/knock-down the mRNA expression of Musa spp. ACC oxidase and inhibit the biosynthesis of ethylene in banana, thereby delaying the ripening of banana, and consequently, prolonging the shelf life of banana.

### BACKGROUND OF THE INVENTION

1. Field of the Invention

The invention relates to a composition and method for prolonging the shelf life of banana by using interfering RNA, and in particular, to a composition and method useful for inhibiting/knocking-down mRNA expression of Musa spp. ACC oxidase gene and further inhibiting/knocking-down the biosynthesis of ethylene by transferring interfering RNA into banana through gene transfer technique.

2. Description of the Prior Art

“Ripening” refers to the self-ripening process that occurs in climacteric fruit and vegetables after picking. For transportation and storage, some climacteric fruit and vegetables need to be picked before they are completely ripened. The reason for early picking is to prolong the transportation and storage period by taking advantage of the after-ripening of climacteric fruit and vegetables themselves. Another method such as low temperatures, air conditioning, ethylene absorption, ethylene inhibitors and the like, can be adopted as required to inhibit/delay the ripening of climacteric fruit and vegetables to achieve the goal of long term storage. If needed to speed up the ripen process in time for bringing the fruit to market, ethylene can be used to promote the ripening of the fruit and vegetables. However, some fruits such as Musa spp. must go through the after-ripening stage in order to be more favorable and edible.

Banana (Musa spp.) is a monocotyledon plant belonging to the Musa genius of Musaceae. Its fruit is fragrant and delicious as well as has high in nutritional value, which makes it economically one of the important crops in the world. Banana is a type of climacteric fruit. This means, that after harvesting, green banana has to undergo climacteric change through its ripening process, including production of internal ethylene, hydrolysis of starch and protopectin, and the like, in order for the fruit flesh to softened, the sweetness to increase, and the fragrance to be produced, and thereby increase its dietary value.

Conventionally, bananas are harvested un-ripened, and are transported and stored in a manner to prolong the banana's ripening process. However, banana fruit may often undergo ripening due to the production of ethylene during the transportation process. Furthermore, the fruit may become over-ripened and spoil, and consequently, the market value of the banana is lowered and the popularization of banana is affected. Accordingly, control on the biosynthesis of ethylene can be used to provide a method to resolve problems such as untimely ripening of banana and the like.

Ethylene is a plant hormone present in gaseous form, which can affect a number of physiological and biochemical reactions in plant (Burg and Burg, 1962). Ethylene plays a important role in the growth, development, and stress-response of plant, for example, when a plant is subjected to a flood, mechanical injury, bacterial infection, aging of leaf and flower, fruit ripening, and the like, it will produce ethylene. The biosynthesis pathway of ethylene comprises the conversion of methionine into S-Adenosyl-methionine (AdoMet) with the aid of AdoMet synthase, synthesis of 1-aminocyclopropane-1-carboxylic acid (ACC) from AdoMet with the aid of ACC synthase (ACS), and then oxidation of ACC into ethylene with the aid of ACC oxidase (ACO) (Yang and Hoffman, 1984). It is known that ACO is the last enzyme used in the biosynthesis pathway of ethylene, and as a result, inhibition on ACO gene or protein expression thereof can inhibit/knock-down the biosynthesis of ethylene, and achieve the object of retarding the after-ripening of a fruit. The invention uses ACC oxidase genes of banana (Musa spp.): Mh-ACO1A (MAO1A, GenBank accession no.: AF030411, SEQ ID No:14), Mh-ACO1B (MAO1B, GenBank accession no.: AF030410, SEQ ID No:15) and Mh-ACO2 (MAO2, GenBank accession no.: U86045, SEQ ID No:16) as target genes, to inhibit/knock-down the biosynthesis of ethylene through inhibiting expression of said genes, thereby achieving the object of retarding the ripening of fruit.

In the past, methods for knocking-down the expression of a target gene consisted mainly of transferring the antisense strand of the target gene into plant, such that the mRNA produced thereof is complementary to the mRNA sequence of an endogenous sense gene, the duplex structure thus formed could then degrade or interfere with the progress of protein translation, and achieve the object of inhibiting the expression of an endogenous gene. Alternatively, construction of a sense strand of a target gene associated with an over-expressing promoter, so that the over-expression of said gene can cause a co-suppression phenomenon and inhibit the expression of said gene. Unfortunately, the effect of silencing genes by the two above-described methods is not good enough. As the gene silencing mechanism has been gradually understood in greater detail, a double-stranded RNA has been considered as the main factor causing the gene silence. As a result, it has been found that constructing a DNA structure capable of forming double-stranded RNA, and transferring it into an organism could enhance the ability of gene silencing, and wherein, if a functionally intact intron was used as a spacer of a loop, the gene expression could be inhibited almost completely (Smith et al., 2000).

RNA interference (RNAi) is a method for knocking-down the expression of target gene. Said method uses small single-stranded or double-stranded RNA (ssRNA or dsRNA) to silence the expression of a gene. Interfering RNA includes small interfering RNA (siRNA), double-stranded or single-stranded RNA (ds siRNA or ss siRNA), microRNA (miRNA), short hairpin RNA (shRNA) and the like. RNA interference will occur within living cells, dsRNA will be recognized by a RNaseIII-like enzymes called dicer, which cuts dsRNA into small RNA molecules with 3′ end having 2-nucleotide overhang, that is siRNA, with a size of about 21 to 23 nucleotides (Elbashir et al., 2001; Zamore et al., 2000).

siRNA can bind with a protein complex. This protein complex is called RNA-induced silencing complex (RISC). RISC has a helicase that can unzip a double-stranded siRNA to form a single-stranded structure, wherein the antisense strand (or guide strand) of siRNA will bind with RISC so as to guide RISC onto the target mRNA, and initiate the degradation of the target mRNA, thereby silencing further expression of the target gene (Matzke et al., 2001; Waterhouse et al., 2001); and wherein said target mRNA is a sequence complementary to the antisense strand of said siRNA.

Smith et al. (2000) had transferred antisense or sense Nia-protease (Pro) gene of potato virus Y (PVY) into potato so as to render potato resistant to PVY The ratio of generating gene-silenced transgenic plant from these two strategies are 7% and 4%, respectively. Nevertheless, if a inverted-repeat DNA capable of forming a double-stranded RNA is used and a functionally intact intron is constructed as a spacer of a loop, the transformation efficiency can be increased up to 96% (22/23). It is suggested that the presence of the intron can facilitate the stability of RNA, adjust the direction of RNA, and a duplex may be formed transiently from a preRNA during the splicing process in eukaryote. This character can be used to facilitate the formation of a double-stranded RNA, thereby increasing the inhibition effect (Smith et al., 2000).

In general, the gene transfer method can be carried out by transforming embryogenic materials such as embryogenic callus, embryogenic suspension, or somatic cell, with Agrobacterium containing an exogenous gene to obtain said transgenic plant. Gene transfer method for banana had been disclosed generally in relative literature or patent for example, S. S. Ma (1988) “Somatic embryogenesis and plant regeneration from cell suspension culture of banana;” or Dean Engler et al. U.S. Pat. No. 6,133,035 titled “Method of genetically transforming banana plants.” Nevertheless, one skilled in the art of this field knows that gene transfer techniques for different species or different genes, may affect the success rate of delivering gene into an organism due to genomes of different species, different gene structure and the like. Furthermore, it is necessary to improve the gene to be transferred or the manner used for delivering the gene in accordance with the specific requirement of different genes or different species. In consideration of this, this application intends to transform RNAi into banana to inhibit/knock-down the genes involved in biosynthesis of ethylene, and achieve the object to prolonging the shelf life of banana fruit.

In view of the importance in the banana industry of keeping fruit fresh and delaying ripening of fruit, the inventor had thought to improve, and finally has successfully developed the composition and method for prolonging the shelf life of banana by using interfering RNA (RNAi) according to the invention.

### SUMMARY OF THE INVENTION

One object of the invention is to provide a composition for prolonging the shelf life of banana by using an interfering RNA, characterized in that said composition comprises an interfering RNA, wherein said interfering RNA is to be transferred into banana by means of gene transfer technique, so as to inhibit/knock-down the biosynthesis of ethylene.

Another object of the invention is to provide a method for prolonging the shelf life of banana by using interfering RNA, characterized in that said method transferes an interfering RNA into banana to inhibit/knock-down the expression of banana ACC oxidase gene, thereby inhibit/knock-down the biosynthesis of ethylene, for prolonging the shelf life of banana.

Yet another object of the invention is to provide a control cassette for controlling banana ACC oxidase, characterized in that said control cassette comprises an interfering RNA to inhibit/knock-down the expression of banana ACC oxidase gene.

Still yet another object of the invention is to provide a novel gene transfer method for banana, characterized in that said gene transfer method comprises of carrying out gene transfer by using callus cell induced from male inflorescence of banana, or somatic embryo cell induced from fruit finger primodia of banana, or somatic embryo cell induced from apical meristem of banana, as the transforming material to obtain transgenic banana.

Yet still another object of the invention is to provide a process for inhibiting/knocking-down the biosynthesis of ethylene in banana, characterized in that the inventive composition for controlling ACC oxidase of banana is transformed into banana by means of the inventive gene transfer method so as to inhibit the mRNA expression of ACC oxidase and hence inhibit/knock-down the biosynthesis of ethylene in banana.

The composition and method for prolonging the shelf life of banana by using interfering RNA that can achieve the above-described objects of the invention comprises:

At least one interfering RNA, a gene transfer expression vector and a pharmaceutically acceptable carrier; wherein said interfering RNA is linked to the 3′ end of the promoter of said gene transfer expression vector, and is constructed in said gene transfer expression vector in accordance with a order as antisense strand—intron—sense strand of banana gene sequence, and wherein said interfering RNA is used to inhibit the gene expression associated with the enzyme involved in the biosynthesis of ethylene in banana;

Wherein said interfering RNA has a sequence as shown in SEQ ID No: 1, can inhibit simultaneously the mRNA expression of ACC oxidase-1A (MAO1A) and ACC oxidase-1B (MAO1B) in banana, and hence knock-down further the quantity of the biosynthesis of ethylene;

Wherein said interfering RNA has a sequence as shown in SEQ ID No: 2 can inhibit the mRNA expression of Musa spp. ACC oxidase-2 (MAO2) in banana, and hence knock-down further the quantity of the biosynthesis of ethylene;

Wherein the sequence of said interfering RNA is constructed in a manner of antisense strand—intron—sense strand; wherein said antisense strand, intron or sense strand compare with the mRNA sequence of banana target gene, with at least 80% sequence complementary, or at least 90% sequence identity;

Wherein said carrier may be water, or various suitable buffer solution, that facilitate said interfering RNA or expression vector thereof easy operation, storage or more stable and not susceptible to degradation.

A banana ACC oxidase control cassette is provided by the invention and comprises:

A above-described interfering RNA; and

A gene transfer expression vector;

Wherein said interfering RNA is linked to the 3′ end of a gene transfer expression vector promoter, said promoter can activate the transcription of said interfering RNA in banana containing said banana ACC oxidase control cassette.

The above-described gene transfer expression vector includes, but not limited to: pBI101, pBI121, pBIN19(ClonTech), pCAMBIA1301, pCAMBIA1305, pGREEN (GenBank Accession No: AJ007829), pGREEN II (GenBank Accession No: EF590266) (www.pGreen.ac.uk), and pGreen0029 (John Innes Centre).

In the process using callus cell induced from male inflorescence of banana as the transforming material according to the invention, the male inflorescence of banana is placed in a suitable medium to induce the formation of callus; after forming the callus, callus cell is taken to form a homogeneously suspension cell in a suitable medium; a transformed Agrobacterium (containing a gene transfer expression plasmid, wherein said plasmid can express the above-described interfering RNA in a plant) is transformed in said callus cell (Agrobacterium mediation method); after a suitable period of culturing, these strains are screened with medium containing suitable antibiotics to select successfully transformed strains; the survived strains are placed in a suitable medium to carry out the differentiation of somatic embryo, and the induction of multiple shoot and root.

The invention further provides the inventive process using fruit finger primodia or apical meristem of banana as the gene transfer materials and comprising:

Placing the fruit finger primodia or apical meristem in a suitable medium to induce the formation of somatic embryo cell; transferring a transformed Agrobacterium (containing a gene transfer expression plasmid, wherein said plasmid can express the above-described interfering RNA in a plant) in said somatic embryo cell (Agrobacterium mediation method); after culturing for a suitable period, screening said strains in a medium containing suitable antibiotics to selected successfully transformed strains; the survived strains are placed in a suitable medium to carry out the induction of multiple shoot and root.

The above-described transfer approaches include, but not limited to: Agrobacterium mediation, genetic recombination virus infection, transposon vector transfer, gene gun transfer, electroporation, microinjection, pollen tube pathway, liposome-mediated transfer, ultrasonic mediation transfer, silicon carbide fiber-mediated transformation, electrophoresis, laser microbeam, polyethylene glycol (PEG), calcium phosphate co-precipitation, DEAE-dextran transformation and the like.

The term “transgenic strain or transformed strain” as used herein refers to a plant strain obtained through transformation such that an exogenous gene is transformed into a target plant, thereby change the genomic constitution of said plant and that the exogenous gene can exist in said target plant and progeny thereof.

The term “gene expression” used herein refers to the expression of mRNA or protein.

These features and advantages of the present invention will be fully understood and appreciated from the following detailed description of the accompanying Drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These color drawings are necessary to fully show the color turning in the fruit ripening of Musa spp.

FIG. 1A-C is the flow chart for constructing universal vector pRNAi used in silencing a gene.

FIG. 2A-F shows the construction strategy for inhibiting gene vectors pBI121-2AnS and pBI121-1AnS associated with the ripening of banana.

FIG. 3 shows the Agrobacterium-mediated gene transfer carried out on banana and the regeneration of the transgenic strain. FIG. 3A: Cell obtained after Agrobacterium-mediated gene transfer is placed on screening medium. FIG. 3B: Cell mass on the medium. FIG. 3C: The growth of somatic embryo. FIG. 3D: The amplified image of the somatic embryo. FIG. 3E: The cultivation on the medium for inducing the formation of plantlet. FIG. 3F: Regenerated plantlet. Bars (A) (D)=1 mm. Bars (B)(C)(E)=5 mm.

FIG. 4 shows the growth of banana strain which has been transformed with vector pBI121-2AnS. FIG. 4A, 4C, 4E, 4G, and 4I show the un-transformed banana strain as control groups. FIG. 4B, 4D, 4F, 4H, and 4J show the transformed banana strain. FIG. 4K shows the fruit of un-transformed banana strain. FIG. 4L shows the fruit of transformed banana strain; no considerable difference in the appearance existed between both strains.

FIG. 5 shows the result of Southern Blot on the genomic DNA of the gene-silenced strain. FIG. 5A: Probes used for the construction and hybridization of T-DNA domain in pBI121-2AnS. FIG. 5B: Result of the Southern Blot obtained by using Mh-ACO2 gene fragment as the probe.

FIG. 6 shows the expression of Mh-ACO2 gene in the Musa spp. cv. Pei Chiao, AAA group transgenic strain that had been transformed with a vector containing silenced banana ripening-associated gene. FIG. 6A shows the result of RT-PCR on leaves of various transgenic strains. FIG. 6B shows the quantitative linear bar chart of RT-PCR expression on various transgenic strains, where the calculation standard was based on the expression quantity of un-transformed control group as 100%, and the result indicated that gene expression of various transgenic strains were inhibited to various level.

FIG. 7 shows the Mh-ACO2 gene expression in various organs of the Musa spp. cv. Pei Chiao, AAA group transgenic strain 2AS-79 that had been transformed with a vector containing silenced banana ripening-associated gene. FIG. 7A shows the RT-PCR analytical result on organs of un-transformed control plant and 2AS-79 transgenic strains. FIG. 7B shows the quantitative linear bar chart of RT-PCR expression on various transgenic strains, where the calculation standard was based on the expression quantity of un-transformed control plant as 100%, and the result indicated that gene expression of various parts on transgenic strains were inhibited to various level.

FIG. 8 shows the siRNA expression in various parts of the Musa spp. cv. Pei Chiao, AAA group transgenic strain 2AS-79 that had been transformed with a vector containing silenced banana ripening-associated gene. The probe used in the hybridization was Mh-ACO2 gene fragment, and synthetic specific Mh-ACO2 gene fragments of 25 nucleotides (nt) and 17 nt in length were used as control groups. WT-O: ovary of un-transformed banana; 79-Pi: pistil of 2AS-79 transgenic strain; 79-S: stamen of 2AS-79; 79-Pe: petal of 2AS-79; 79-O: ovary of 2AS-79; 79-B: bract of 2AS-79.

FIG. 9 shows the color turning in the fruit ripening of Musa spp. cv. Pei Chiao, AAA group that had been transformed with a vector containing silenced banana after-ripening-associated gene. FIG. 9A: the process of pericarp color turning. FIG. 9B: pericarp color index chart. Bars=5 cm.

FIG. 10 shows the change of respiration rate and producing quantity of ethylene in the fruit ripening of Musa spp. cv. Pei Chiao, AAA group that had been transformed with a vector containing silenced banana ripening-associated gene. FIG. 10A: Change of respiration rate in the course of fruit ripening. FIG. 10B: Change of producing quantity of ethylene in the course of fruit ripening.

FIG. 11 shows the color turning of pericarp after ripening treatment on fruit of Musa spp. cv. Pei Chiao, AAA group that had been transformed with a vector containing silenced banana ripening-associated gene. FIG. 11A: Color turning course of pericarp. FIG. 11B: color index chart of pericarp. Bars=5 cm.

FIG. 12 shows the change of respiration rate and producing quantity of ethylene after ripening treatment on the fruit of Musa spp. cv. Pei Chiao, AAA group that had been transformed with a vector containing silenced banana ripening-associated gene. FIG. 12A: Change of respiration rate after ripening treatment on fruit. FIG. 12B: Change of producing quantity of ethylene after ripening treatment on fruit.

### DETAILED DESCRIPTION OF THE PREFERRED EMBODIMENT

The invention will be illustrated more detailed with the following examples, but the invention is not limited thereto.

### EXAMPLE 1

### The Construction of Vector Containing Inhibited Banana Ripening-Associated Gene

1. Construction of Universal Vector pRNAi Containing Silenced Gene

### (1) Plasmid Material

a. pUC19 plasmid: with a total length of 2,682 kb, containing Escherichia coli screening gene AmpR (GenBank accession no. L09137).
b. pU35STgfp plasmid: containing 2× En CaMV 35S promoter, green fluorescent protein (GFP) gene, and nopaline synthase (NOS) gene terminator. The source of said primary plasmid was referenced to PNAS (1996) 93:5888-5893, from which 1,854 bp was cleaved by single digestion with restriction enzyme HindIII, and ligated into vector pUC19 that had been single digested with HindIII to form pU35STgfp.

### (2) Gene Sources

The source of intron used in the construction of vector pRNAi was the first intron of banana (Musa spp.) ACC oxidase gene MAO1A (GenBank accession no. AF030411) and of gene MAO1B (GenBank accession no. AF030410) (with a sequence as shown in SEQ ID No:17). The first intron of both two genes has an identical sequence.

### (3) The Extraction of Banana (Musa spp.) Genomic DNA

1 g samples was cut off, ground in liquid nitrogen, and was added 15 ml extraction buffer (100 mM Tris-HCl, pH 8.0; 50 mM EDTA; 500 mM NaCl), and 1 ml 20% SDS thereto. After standing at 65° C. for 10 minutes, 5 ml 5 M KOAc was added and the resulted mixture was stood on ice for 20 minutes. The mixture was centrifuged at 25,000 xg and 4° C. for 20 minutes. The supernatant was filtered through nylon mesh. 10 ml isopropanol was added to the filtrate to allow precipitating for 30 minutes. The mixture was centrifuged at 4° C. and 20,000 xg for 15 minutes. The supernatant was discarded, and the pellet was air dried. 0.7 ml High TE (50 mM Tris-HCl, pH 8.0; 10 mM EDTA) was added to dissolve the pellet, 75 μl 3M NaOAc and 500 μl isopropanol was added and mixed well. The mixture was centrifuged at 4° C. with microcentrifuge for 10 minutes. The pellet was washed to remove salt with 70% and 100% ethanol, respectively, and then air dried, dissolved in 100 μl TE (pH 8.0) and stored for later use.

### (4) Construction Scheme

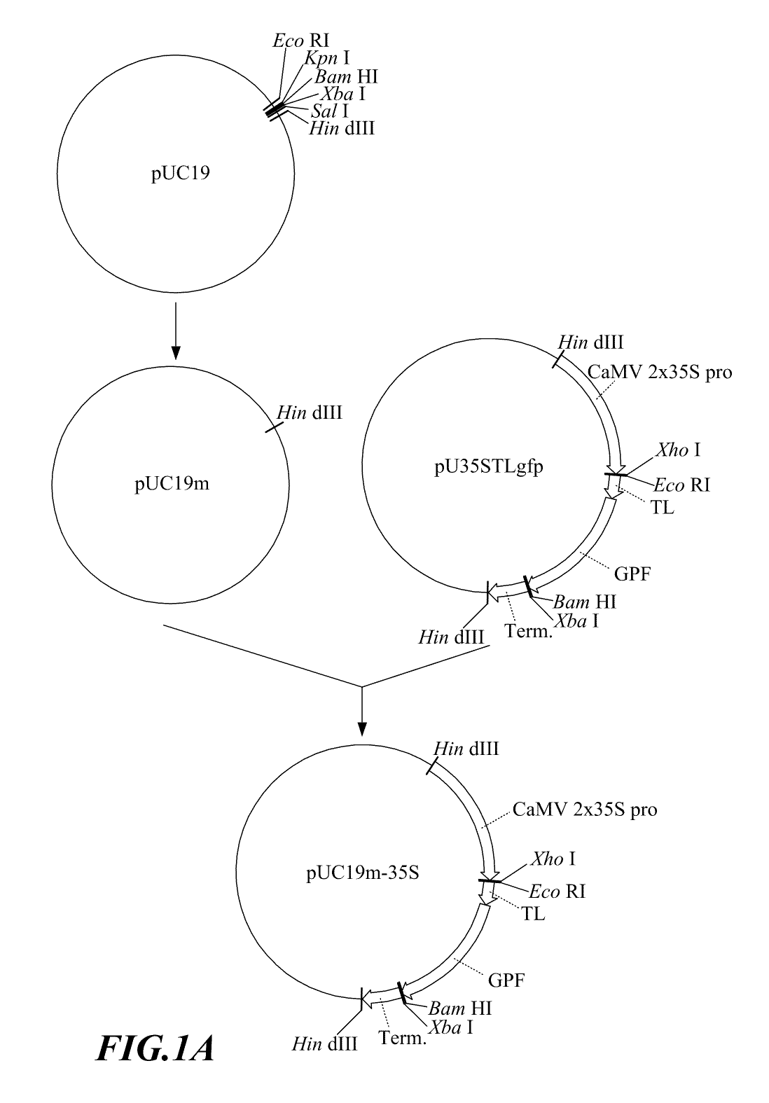

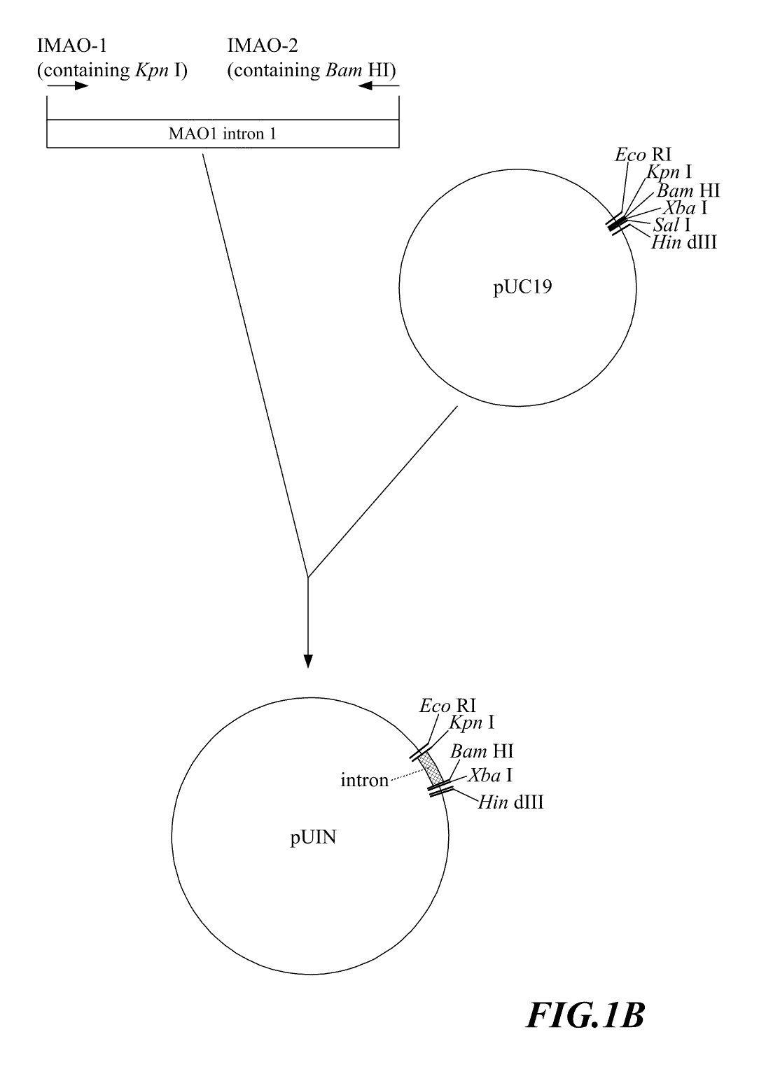

As shown in FIG. 1A, at first, pUC19 was digested with EcoR I and Sal I to remove most of Multiple cloning site (MCS) on pUC19, and then was subjected to blunt end treatment with Klenow enzyme. Then, the product was separated by electrophoresis, and a fragment of 2.6 kb was recovered. Thereafter, this fragment was subjected to self-ligation to obtaine an intermediate vector pUC19m. This intermediate vector pUC19m was digested with restriction enzyme HindIII, and a fragment of about 2.6 kb in length was recovered. Separately, pU35STLgfp was digested with restriction enzyme HindIII, and a fragment of 1.8 kb was recovered. The two fragments were ligated. The resulted plasmid was screened further by restriction enzyme and electrophoresis to obtain an intermediate vector pUC19m-35S. Referring to FIG. 1B, banana (Musa spp.) genomic DNA was used as a template to synthesize the first intron of banana (Musa spp.) ACC oxidase gene MAO1 through PCR. The oligonucleotide primers used in the synthesis of said first intron were described as followed:

Forward primer MAO2 5L: (containing the BamHI
restriction site)

(SEQ ID No: 5)

5′-tat [symbol] gagttcgccaacaaag-3′
BamHI
Reverse primer MAO2 3L: (containing the EcoRI
restriction site)

(SEQ ID No: 6)

5′-cca [symbol] gccttcctatactg-3′
EcoRI

The DNA fragment synthesized by IMAO-1 and IMAO-2 primers through PCR was digested with restriction enzymes KpnI and BamHI, and recovered a fragment about 0.12 kb (MAO1 intron1). This fragment was ligated with the digested pUC19 (digested with restriction enzymes KpnI and BamHI) to obtain an intermediate plasmid pUIN of 2.8 kb. As shown in FIG. 1C, said intermediate plasmid pUC19m-35S was digested with restriction enzymes EcoRI and XbaI, and a fragment of 3.6 kb was recovered. Separately, the intermediate plasmid pUIN was digested with restriction enzymes EcoRI and XbaI to cleave the first intron of MAO1 gene, and a fragment of 0.13 kb was recovered. The two fragments obtained above (the first intron fragment of MAO1 gene, and the digested pUC19m-35S) were ligated to obtain RNA-silenced universal vector pRNAi (FIG. 1C).

### 2. The Construction of RNA Silencing Structure for Silencing the Expression of Banana (Musa spp.) MAO2

At first, all RNA of banana (Musa spp.) was extracted by the following process: plant materials were cut and ground in liquid nitrogen into powder. 20 mL 65° C. extraction buffer (2 M NaCl, 25 mM EDTA, pH 8.0, 100 mM Tris-HCl, spermidine 0.5 g/L, 3% Hexadecyl trimethyl-ammonium bromide, 3% polyvinyl-pyrrolidone-40, 0.4% 2-mercaptoethanol) was added, stirred homogeneously with a homogenizer and treated at 65° C. for 10 minutes. Equal amount of CI (chloroform: isoamyl alcohol=49:1) was added, mixed homogeneously and then centrifuged. The supernatant was extracted once again. 1/3-fold volume of 8 M LiCl was added, and stood at 4° C. for precipitating overnight. Then, centrifuged at 4° C. and discarded the supernatant. 0.5% SDS was used to suspend RNA. Equal volume of CI was added and mixed by shaking for several seconds. After centrifuged at 4° C., 2-fold volume of 100% ethanol was to the supernatant, and the mixture was placed at −20° C. for precipitating. Thereafter, the mixture was centrifuged at 4° C. and the supernatant was discarded. 500 μL of 70% ethanol was added to the residue, the resulted mixture was centrifuged at 4° C. and the supernatant was discarded. 500 μL of 100% ethanol was added, centrifuged at 4° C. and the supernatant was discarded. The RNA precipitate was air dried. The RNA was dissolved in a suitable amount of DEPC-treated water, the concentration of the solution was determined and the solution was stored for later use.

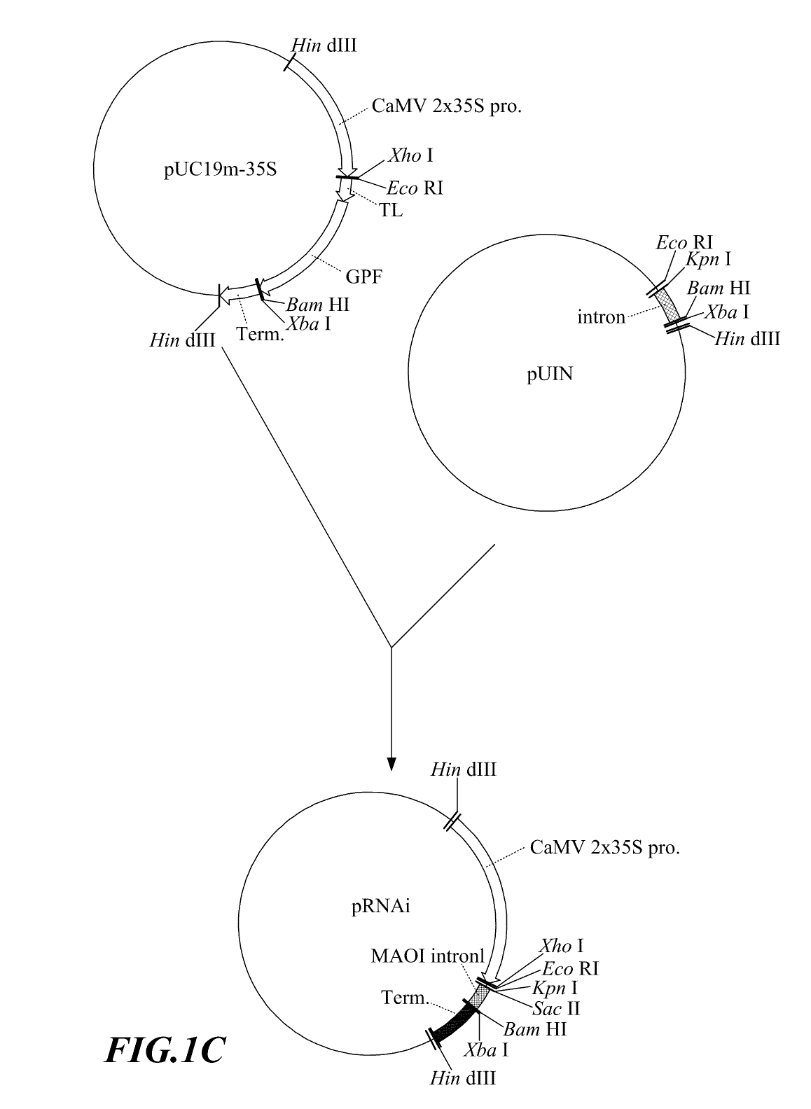

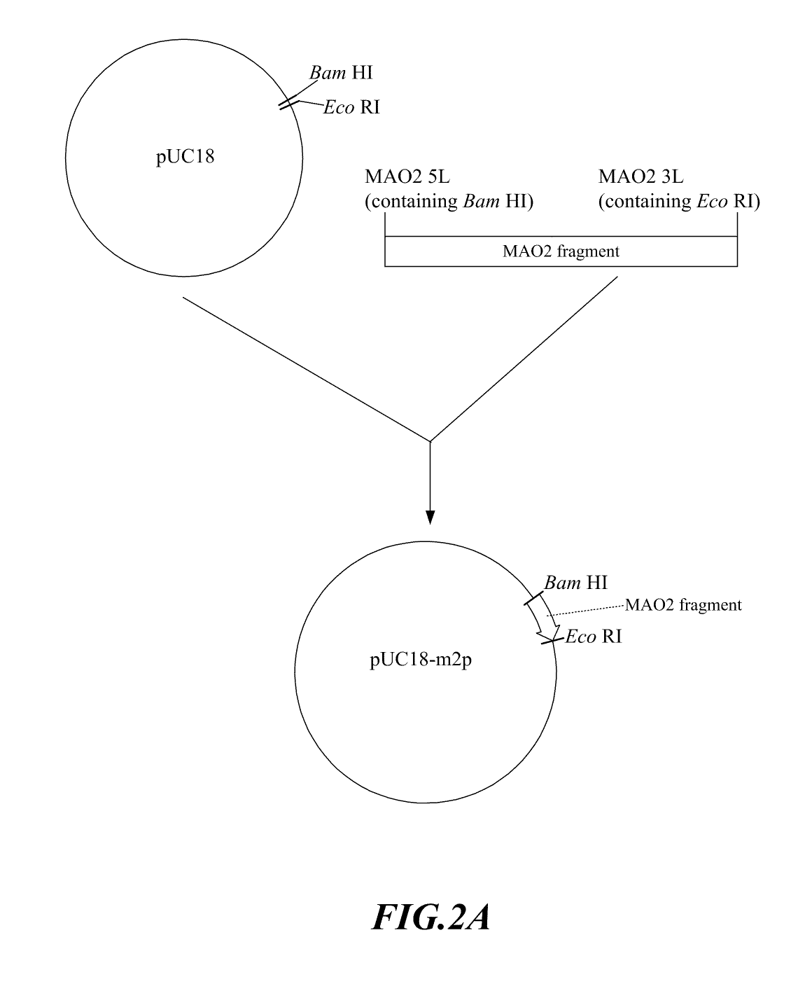

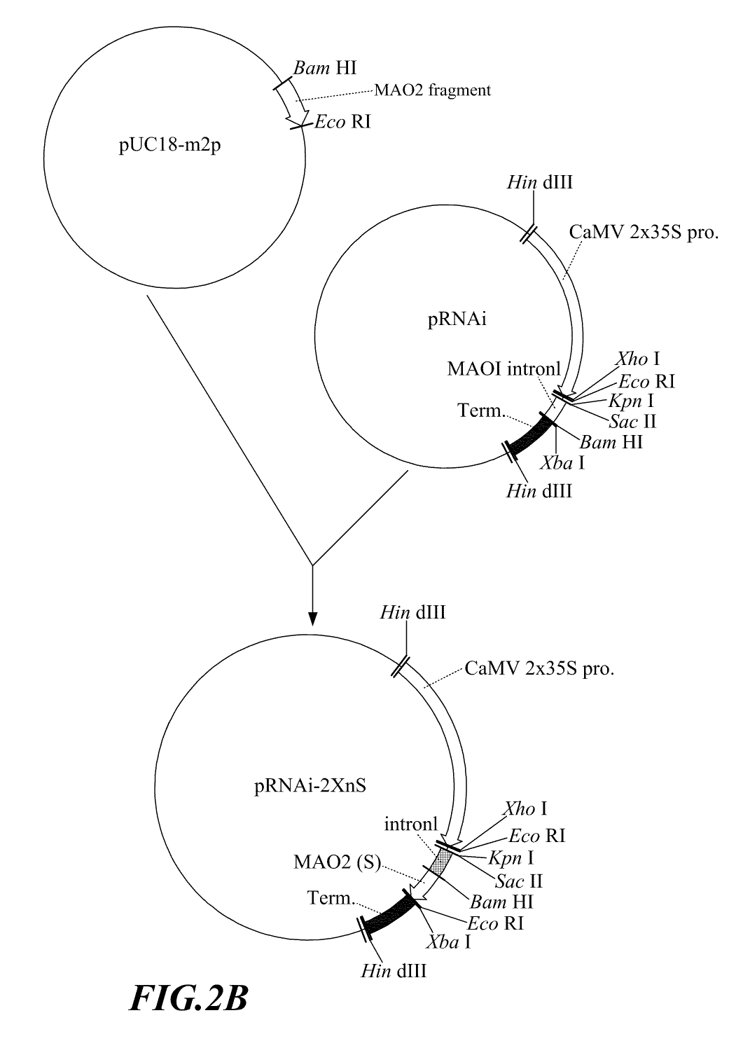

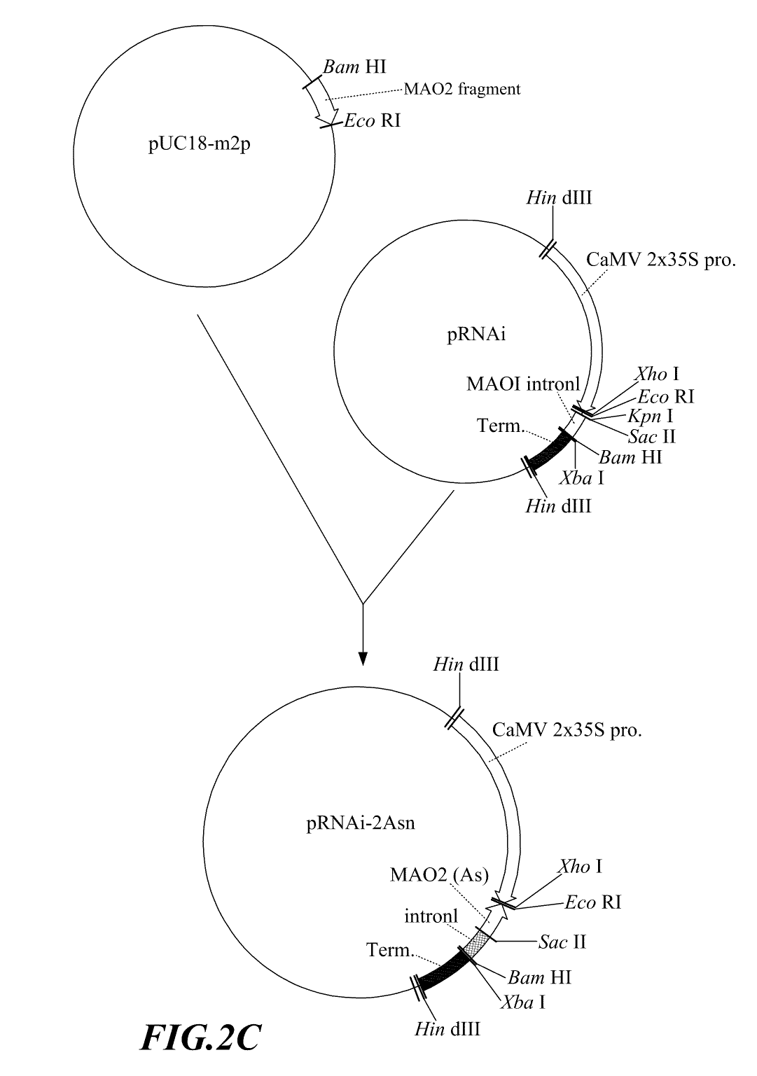

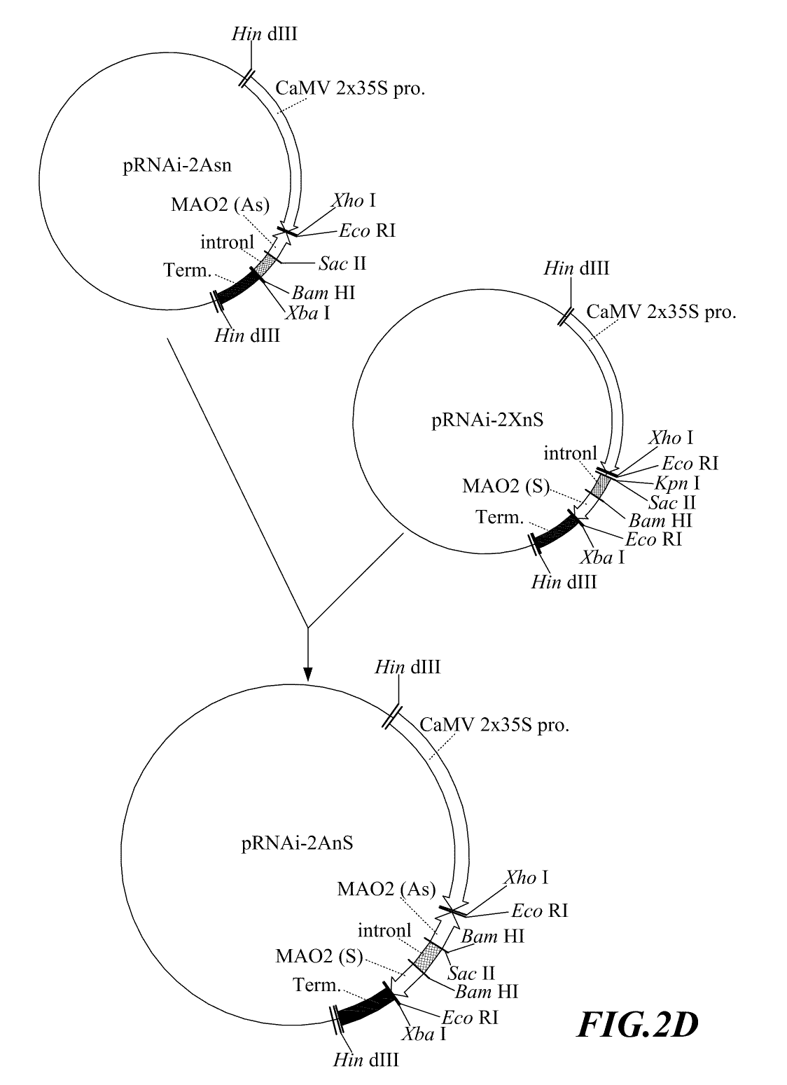

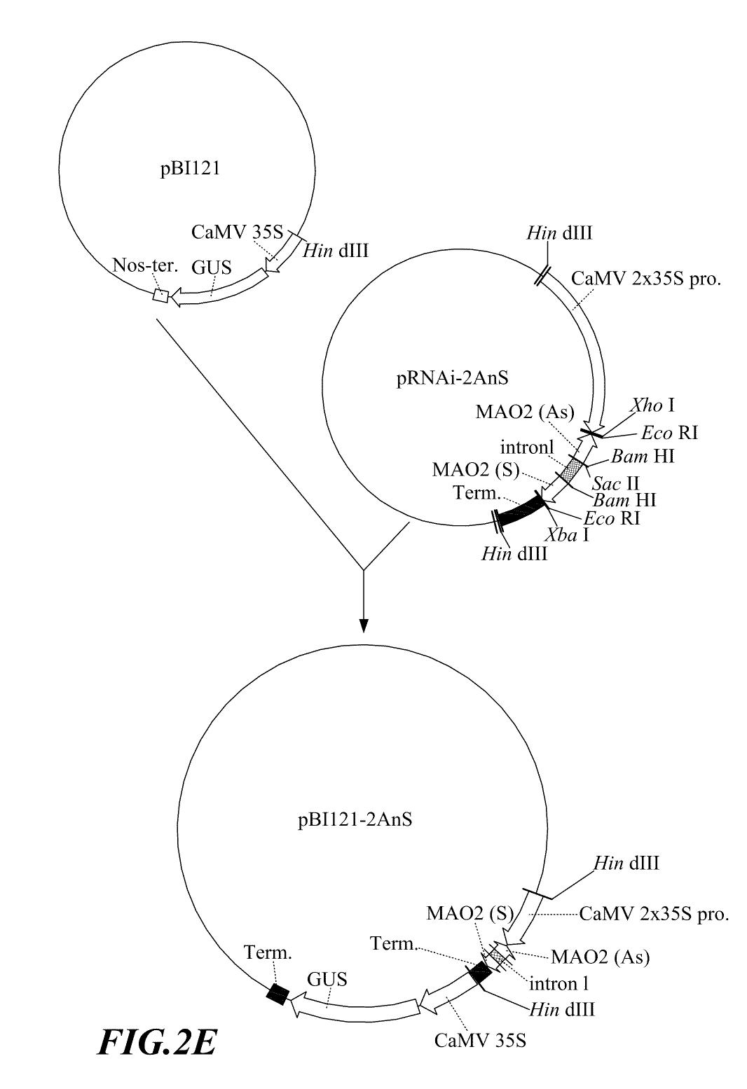

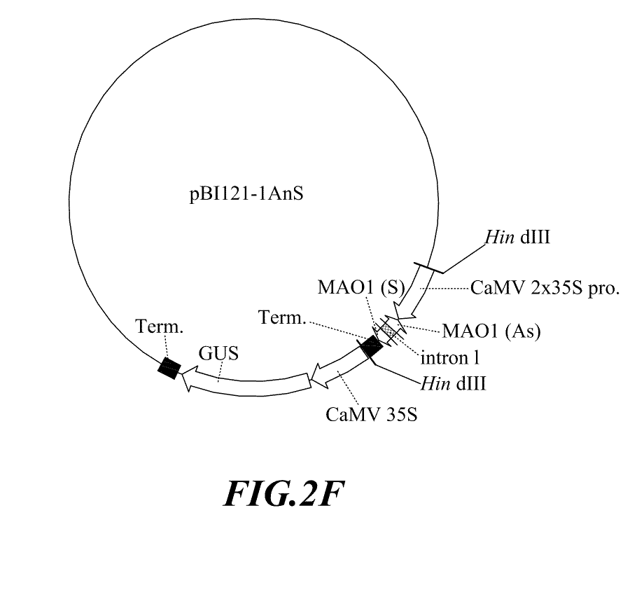

The construction scheme of pRNAi-2AnS plasmid (antisense-sense) was carried out with reference to FIG. 2:

### Step 1:

Referring to FIG. 2A, at first, the total RNA of banana (Musa spp.) was used as the template, a reaction was carried out by using One-Step RT-PCR Kit (GeneMark). The reaction solution comprised 0.1 g/L of template RNA, 50 ng/L of primers, 1× Reaction Mix, 1× Enhancer, 2% Enzyme Mix. The reaction was carried out at a temperature of 50° C. for 30 minutes, 94° C. for 2 minutes, and then 35 cycles of at 94° C. for 30 seconds, 59° C. for 30 seconds, and 72° C. for 1 minute. Finally, reacted at 72° C. for 10 minutes, and then stored at 4° C. for later use. Primers used therein were as followed:

Forward primer MAO1 5L: (containing the BamHI
restriction site)

(SEQ ID No: 7)

5′-ata [symbol] aaacccgttcag-3′
Bam HI

Reverse primer MAO1 3L: (containing the EcoRI
restriction site)

(SEQ ID No: 8)

5′-cat [symbol] gtctcctcgaagtccg-3′

A nucleotide fragment (MAO2 fragment) of about 0.14 bp in length was synthesized by PCR. Said MAO2 fragment was digested with BamHI and EcoRI and product was recovered. Said MAO2 fragment was ligated with the digested pUC18 (digested with BamHI and EcoRI) to obtained a plasmid pUC18-m2p containing MAO2 cDNA of 139 bp (FIG. 2A).

### Step 2:

The plasmid pRNAi was digested with XbaI, then was blunt end treated with Klenow enzyme, digested with BamHI, and a fragment of 3.8 kb was recovered. pUC18-m2p obtained in step 1 was digested with EcoRI, subjected to blunt end treatment with Klenow enzyme, digested with BamHI, and a fragment of 0.14 kb was recovered. After recovering the two above-described digested DNA fragment (pUC18-m2p and pRNAi), they were subjected to ligation, and recovered a plasmid pRNAi-2xnS containing a cDNA fragment of part sense MAO2 (FIG. 2B).

### Step 3:

The plasmid pRNAi was digested with KpnI, blunt end treated with Klenow enzyme, digested with EcoRI, and recovered a fragment of 3.8 kb. Separately, pUC18-m2p obtained in step 1 was digested with BamHI, blunt end treated with Klenow enzyme, digested with EcoRI, and recovered a fragment of 0.14 kb. The two above-described digested and recovered DNA fragment (pUC18-m2p and pRNAi) were subjected to ligation to obtain an intermediate plasmid pRNAi-2Asn containing a cDNA fragment of antisense MAO2 (FIG. 2C).

### Step 4:

Both the pRNAi-2Asn obtained in step 3 and the pRNAi-2xnS obtained in step 2 were digested with XhoI and SacII, and recovered fragments of 150 bp and 3.8 kb, respectively. After these two fragments were subjected to ligation, a plasmid pRNAi-2AnS containing cDNA sequence of part MAO2: antisense MAO2 (antisense strand MAO2 fragment sequence as shown in SEQ ID No:18), and sense MAO2 (sense MAO2 fragment sequence as shown in SEQ ID No:19) (pRNAi-2AnS possessed a constructed sequences in following order (5′ end to 3′ end): antisense MAO2-first intron-sense MAO2, as shown in SEQ ID No:2) (FIG. 2D).

### Step 5:

The plasmid pRNAi-2AnS was digested with HindIII, and recovered a fragment of 1.4 kb. This fragment was ligated with HindIII-digested pBI121 (GenBank accession no. AF485783) and obtained a plasmid pBI121-2AnS to be used in Agrobacterium-mediated transfer (FIG. 2E).

3. The Construction of RNA Silencing Structure for Silencing the Expression of Musa spp. MAO1

The construction scheme of pRNAi-1AnS plasmid (antisense-sense) was carried out similar to the construction strategy of MAO2, except that the primers used in the PCR screening of MAO1 fragment were different as followed:

| | |
|---|---|
| Mh-ACO2 gene: | |
| Forward primer MAO2-5RT: | |
| 5′-atggattcctttccggttatcgaca-3′ | (SEQ ID No: 10) |
| Reverse primer MAO2-3RT: | |
| 5′-attccttcatcgccttccta-3′ | (SEQ ID No: 11) |
| Banana (Musa spp.) actin gene: | |
| Forward primer BACT5: | |
| 5′-tagcggacgtaccacaggtat-3′ | (SEQ ID No: 12) |
| Reverse primer BACT3: | |
| 5′-gtaagcaagcttctccttgat-3′ | (SEQ ID No: 13) |

Referring to the construction strategy of MAO2, a plasmid pBI121-1AnS containing a cDNA sequence of part MAO1: antisense MAO1 (antisense strand MAO1 fragment sequence as shown in SEQ ID No:20), and sense MAO1 (sense strand MAO1 fragment sequence as shown in SEQ ID No:21) (the plasmid pBI121-1AnS possessed a constructed sequences in following order (5′ end to 3′ end): antisense MAO1-first intron-sense MAO1, as shown in SEQ ID No:1; since MAO1A and MAO1B of banana (Musa spp.) possessed highly conserved sequences, the inventive MAO1 interfering RNA contained the above-constructed sequence as shown in SEQ ID No:1 and could inhibit simultaneously gene expression of both MAO1A and MAO1B) (FIG. 2F).

The above-described example illustrates only a preferred embodiment of the invention, is not intended to limit the construction manner of the invention, and other suitable construction strategy is also included within the scope of the invention.

### EXAMPLE 2

### Gene Transfer Technique and Scheme for Banana (Musa spp.)

### 1. Agrobacterium-Mediated Gene Transfer

Both of A and B gene transfer processes for banana (Musa spp.) were those modified from Ma (1988), and comprised the following processes using the below materials and steps:

### Process A

### (1) Plant Materials and Other Materials

Banana strain was Musa spp. cv. Pei Chiao, AAA group strain. Cell suspension was obtained through the induction of male inflorescence, and callus thereof. Following materials were commercially available.

### (2) The Mediator Used for Transfer

The strain of Agrobacterium used in this example was LBA4404 (Hoekema et al., 1983), which was used for the transformation (see Molecular Cloning) of pBI121-2AnS or pBI121-1AnS plasmid constructed in example 1.

### (3) Gene Transfer Method for Banana (Musa spp.)

### Step 1: The Induction of Callus

Male inflorescence of Musa spp. cv. Pei Chiao, AAA group strain was placed on an induction medium (callus-inducing medium, as shown in Table 1) to induce the formation of callus.

TABLE 1
The composition of callus-inducing medium

| Ingredients | Concentration | |
|---|---|---|
| MS salt | 1X | |
| Thiamine-HCl | 0.1~1 | mg/L |
| nicotinic acid | 0.5 | mg/L |
| pyridoxine-HCl | 0.5 | mg/L |
| glycine | 2 | mg/L |
| myo-inositol | 100 | mg/L |
| Biotin | 1 | mg/L |
| IAA | 1 | mg/L |
| NAA | 1 | mg/L |
| 2.4-D | 4 | mg/L |

| | |
|---|---|
| Sucrose | 3~4.5% |
| Agar | 0.7% |

Note 1:
Wherein 0.7% Agar might be 0.2%~0.3% Gelrite.
Note 2:
The final pH value of the medium was pH 5.3~5.7.

### Step 2: Preparation of Cell Suspension

After the callus was formed, a suitable quantity of callus cell was placed in a suspension medium (Table 2) and the callus cell was suspended to form homogeneous cell suspension.

TABLE 2
Composition of suspension medium

| Ingredients | Concentration | |
|---|---|---|
| MS salt | 1X | |
| Thiamine-HCl, | 0.1~1 | mg/L |
| nicotinic acid, | 0.5 | mg/L |
| pyridoxine-HCl, | 0.5~5 | mg/L |
| glycine, | 2~5 | mg/L |
| myo-inositol | 100 | mg/L |
| Biotin | 1 | mg/L |
| glutamine | 0~100 | mg/L |
| malt extract | 0~500 | mg/L |
| proline | 0~230 | mg/L |
| Picloram | 0~1 | mg/L |
| 2.4-D | 1 | mg/L |

| | |
|---|---|
| Sucrose | 3~4.5% |

Note 1:
Wherein 1 mg/L 2.4-D might be a hormone mix containing 1 mg/L IAA, 1 mg/L NAA and 4 mg/L 2.4-D.
Note 2:
The final pH value of the medium was pH 5.3~5.7.

Step 3: The Incubation (or Cocultivation) with Agrobacterium

Before gene transferring, a monocolony of transformed Agrobacterium was inoculated in 20 ml YEB liquid medium (5 g/l beef extract, 1 g/l yeast extract, 5 g/l pepton, 5 g/l manitol, 0.5 g/l MgSO₄, pH 7.5, 12.5 g/l agar) supplemented with proper quantity of antibiotics (50 μg/ml kanamycin, 20 μg/ml stryptomycin and 100 μg/ml Rifamycin) and cultured by shaking at 28° C. and 240 rpm for 2 days. As OD₆₀₀ was 1.0˜1.5, the bacteria liquor was centrifuged at 4,000 rpm(HERMLE Z363 K) for 20 minutes. The supernatant was discarded, and pellet was suspended in a co-culture transferring medium (Table 3) to obtain a bacteria liquor of the transformed Agrobacterium for later use. Said transformed Agrobacterium contained the above-constructed pBI121-2AnS plasmid or pBI121-1AnS plasmid.

A proper quantity of callus cell or its cell suspension was mixed with the bacterial suspension and was co-cultured by shaking, and then by stood at 25° C. for 2-4 days.

TABLE 3
The composition of co-culture transferring medium

| Ingredients | Concentration | |
|---|---|---|
| MS salt | 1X | |
| Thiamine-HCl, | 0.1~1 | mg/L |
| nicotinic acid, | 0.5 | mg/L |
| pyridoxine-HCl, | 0.5 | mg/L |
| glycine, | 2 | mg/L |
| myo-inositol | 100 | mg/L |
| Biotin | 1 | mg/L |
| glutamine | 100 | mg/L |
| malt extract | 500 | mg/L |
| proline | 230 | mg/L |
| 2,4-D | 1 | mg/L |
| betaine | 1 | mM |
| acetosyringone | 0.1~0.3 | mM |

| | |
|---|---|
| Sucrose | 3~4.5% |

Note 1:
Wherein MS salt might be SH salt.
Note 2:
The final pH of the medium was pH 5.3~5.7.

Step 4: Screening after Transferring

After co-culturing for 2-4 days, the thus co-cultured post-transfer cells were placed in a solid post-transfer screening medium (Table 4) to carry out screening operation on transgenic strains.

TABLE 4
The composition of post-transfer screening medium

| Ingredients | Concentration | |
|---|---|---|
| MS salt | 1X | |
| Thiamine-HCl, | 0.1~1 | mg/L |
| nicotinic acid, | 0.5 | mg/L |
| pyridoxine-HCl, | 0.5 | mg/L |
| glycine, | 2 | mg/L |
| myo-inositol | 100 | mg/L |
| Biotin | 1 | mg/L |
| glutamine | 100 | mg/L |
| malt extract | 100~500 | mg/L |
| proline | 230 | mg/L |
| 2.4-D | 1 | mg/L |
| Sucrose | 3~4.5% | |
| Lactose | 0~0.1% | |
| Agar | 0.7% | |
| Cefotaxime | 200 | mg/L |

| | |
|---|---|
| G418 | Suitable |
| | concentration |

Note 1:
Wherein 1 mg/L 2.4-D might be a hormone mixture containing 0.05 mg/L Zeatin, 0.2 mg/L 2-ip, 0.1 mg/L kinetin and 0.2 mg/L NAA.
Note 2:
Wherein 0.7% Agar might be 0.2%~0.3% Gelrite.
Note 3:
Wherein the suitable concentration of G418 might be suitable concentration of hygromycin; wherein said suitable concentration is referred to the concentration used in the screening operation at from low to high stringency, and one preferred example was 50 mg/L G418.
Note 4:
The final pH value of the medium was pH 5.3~5.7.

### Step 5: The Differentiation of Embryo

After culturing in the solid post-transfer screening medium for two months, cells were cultured continuously by changing into regeneration medium (Table 5) till the formation of embryo.

TABLE 5
The composition of regeneration medium

| Ingredients | Concentration | |
|---|---|---|
| MS salt | 1X | |
| Thiamine-HCl, | 0.1~1 | mg/L |
| nicotinic acid, | 0.5 | mg/L |
| pyridoxine-HCl, | 0.5 | mg/L |
| glycine, | 2 | mg/L |
| myo-inositol | 100 | mg/L |
| Biotin | 1 | mg/L |
| glutamine | 0~100 | mg/L |
| malt extract | 0~100 | mg/L |
| Hormone mixture | | |

| | |
|---|---|
| Sucrose | 3~4.5% |
| Lactose | 0~0.1% |
| Agar | 0.7% |
| G418 | Suitable |
| | concentration |

Note 1:
Wherein MS salt might be SH salt, or B5 salt.
Note 2:
Wherein said hormone mixture might be: (1) 0.05 mg/L Zeatin, 0.2 mg/L 2-ip, 0.1 mg/L kinetin and 0.2 mg/L NAA; or (2) 1 mg/L BA and 0.1 mg/L GA.
Note 3:
Wherein 0.7% Agar might be 0.2%~0.3% Gelrite.
Note 4:
Wherein suitable concentration of G418 might be suitable concentration of hygromycin; wherein said suitable concentration is referred to the concentration used in the screening operation at from low to high stringency, and one preferred example was 100 mg/L G418.
Note 5:
The final pH value of the medium was pH 5.3~5.7.

### Step 6: The Induction of Multiple Shoot

After an embryo was formed from cells, the somatic embryo cell was shifted into multiple shoot inducing medium (Table 6) to induce the germinating of multiple shoot from the embryo and then the growth of seedling.

TABLE 6
The composition of multiple shoot-inducing medium

| Ingredients | Concentration | |
|---|---|---|
| MS salt | | |
| Thiamine-HCl, | 0.1~1 | mg/L |
| nicotinic acid, | 0.5 | mg/L |
| pyridoxine-HCl, | 0.5 | mg/L |
| glycine, | 2 | mg/L |
| myo-inositol | 100 | mg/L |
| Biotin | 1 | mg/L |
| Glutamine | 0~100 | mg/L |
| Hormone mixture | | |

| | |
|---|---|
| Sucrose | 3% |
| Agar | 0.7% |
| G418 | Suitable |
| | concentration |

Note 1:
Wherein MS salt might be ½ MS salt, or B5 salt.
Note 2:
Wherein said hormone mixture might be: (1) 1 mg/L BA, 0.1 mg/L GA; or (2) 1 mg/L 2iP, 0.1 mg/L GA.
Note 3:
Wherein 0.7% Agar might be 0.2%~0.3% Gelrite.
Note 4:
Wherein suitable concentration of G418 might be suitable concentration of hygromycin; wherein said suitable concentration is referred to the concentration used in screening operation at from low to high stringency, and one preferred example was 100 mg/L G418.
Note 5:
The final pH value of the medium was pH 5.3~6.0.

### Step 7: The Induction of Root

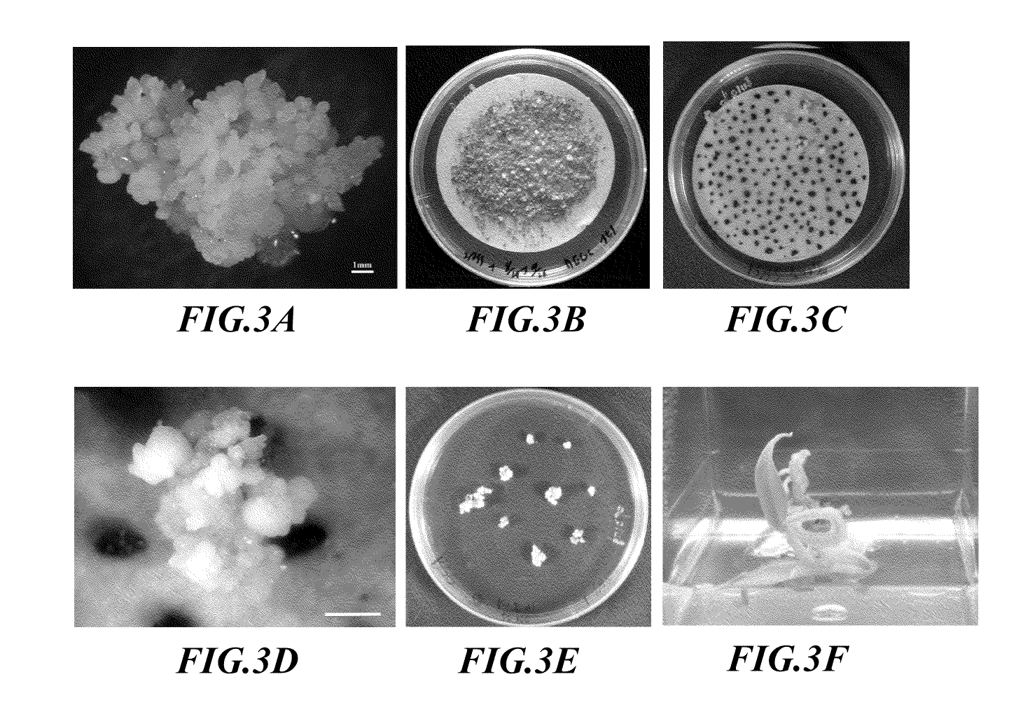

As the seedling had grown to a suitable size, it was shifted to a root-inducing medium (Table 7) to induce rooting, and promote the growth of the plant. (FIG. 3A-F)

TABLE 7
The composition of root-inducing medium

| Ingredients | Concentration | |
|---|---|---|
| MS salt | 1X | |
| Thiamine-HCl, | 0.1~1 | mg/L |
| nicotinic acid, | 0.5 | mg/L |
| pyridoxine-HCl, | 0.5 | mg/L |
| glycine, | 2 | mg/L |
| myo-inositol | 100 | mg/L |
| IBA | 2.5 | mg/L |
| BA | 2.5 | mg/L |

| | |
|---|---|
| Sucrose | 3% |
| Agar | 0.7% |
| G418 | Suitable |
| | concentration |

Note 1:
Wherein 0.7% Agar might be 0.2%~0.3% Gelrite.
Note 2:
Wherein suitable concentration of G418 might be suitable concentration of hygromycin; wherein said suitable concentration is referred to the concentration used in the screening operation at form low to high stringency, and one preferred example was 100 mg/L G418.
Note 3:
The final pH value of the medium was pH 5.3~6.0.

### Process B

### (1) Plant Materials and Other Materials

Banana strain was Musa spp. cv. Pei Chiao, AAA group strain. Following materials were commercially available.

### (2) Mediator for Transfer

Agrobacterium strain, LBA4404 (Hoekema et al., 1983) was used to transformed the above-constructed pBI121-2AnS plasmid.

### (3) Gene Transfer Method for Banana (Musa spp.)

### Step 1: Induction of Embryo

Fruit finger primodia or apical meristem of banana (Musa spp.) was used as the transfer material. The fruit finger primodia or apical meristem was placed in an induction medium (Table 8) to induce the formation of somatic embryo cell.

Step 2: Cocultivation with Agrobacterium

Somatic embryo cell was cocultivation with the above-described transformed Agrobacterium liquor (said transformed Agrobacterium contained the above-constructed pBI121-2AnS plasmid or pBI121-1AnS plasmid) in an induction medium (Table 8).

### Step 3: Post-Transfer Screening

After cocultivation, the transgenic plant was shifted in an induction medium (Table 8) supplemented with 50 mg/L G418 to carry out post-transfer screening.

### Step 4: Culturing Adult-Plant

The thus-screened transgenic plant was shifted in an induction medium (Table 8) containing antibiotics. A transgenic plant could leave the bottle eight months after transfer treatment.

TABLE 8
The composition of induction medium

| Ingredients | Concentration | |
|---|---|---|
| MS salt | 1X | |
| Thiamine-HCl, | 0.1~1 | mg/L |
| nicotinic acid, | 0.5 | mg/L |
| pyridoxine-HCl, | 0.5 | mg/L |
| glycine, | 2 | mg/L |
| myo-inositol | 100 | mg/L |
| IBA | 2.5 | mg/L |
| BA | 2.5 | mg/L |

| | |
|---|---|
| Sucrose | 3% |
| Agar | 0.7% |
| G418 | Suitable |
| | concentration |

Note 1:
Wherein 0.7% Agar might be 0.2%~0.3% Gelrite.
Note 2:
Wherein suitable concentration of G418 might be suitable concentration of hygromycin; and wherein said suitable concentration was referred to the concentration used in the screening operation at from low to high stringency, and one preferred example was 100 mg/L G418.
Note 3:
The final pH value of the medium was pH 5.3~6.0.

### 2. Screening and Growth of Transgenic Musa spp.

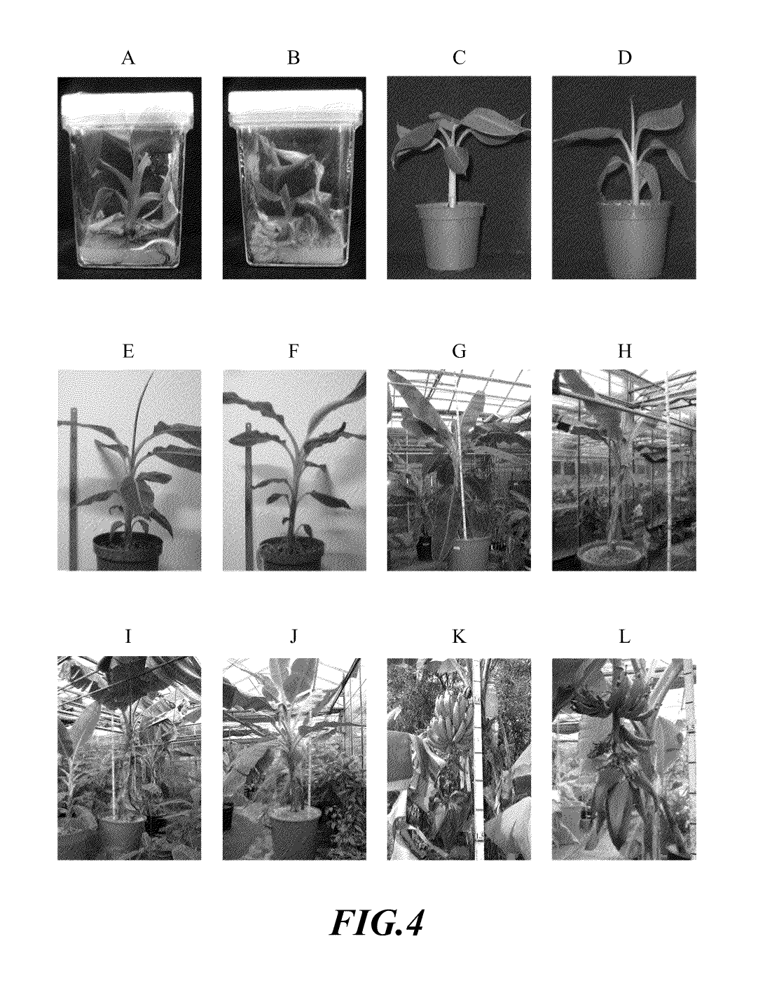

The growth of Mh-ACO2-silenced Musa spp. cv. Pei Chiao, AAA group transgenic plant during the tissue culturing period of the transgenic plant, between field planting and growing to a height of about 1.5 meter, indicated no considerable difference compared with un-transformed Musa spp. cv. Pei Chiao, AAA group control plant (FIG. 4A-H). After cultivating continuously for 5 months, the un-transformed control plant had grown to a height of more than 3 meters, with about 10 health leaves, while the transgenic plant had grown to a height of about 2.5 meters, while number of health leaves was similar to that of control plant, i.e. about 10 leaves (FIG. 4I-L).

### EXAMPLE 3

### Obtaining Molecular Evidence of Transgenic Musa spp. by Southern Blot Analysis

The transgenic Musa spp. cell was screened with antibiotics to regenerate a plantlet, which was subjected to histochemical staining of GUS to identify the reporter gene, and was then subjected to molecular level analysis. In this example, the Southern hybridization analysis was used to confirm that the DNA fragment to be transformed was integrated in the Musa spp. genome.

20 μg plant genomic DNA was digested with suitable restriction enzyme, and was separated then by electrophoresis on 0.7% agarose gel. The electrophoresis gel was treated twice with 0.25 N HCl for 15 minutes, treated twice in a denaturing buffer (1.5 M NaCl, 0.5 M Tris-HCl, pH 7.2, 1 mM Na₂EDTA) for 15 minutes, and then twice in neutralization buffer (1.5 M NaCl, 0.5 M Tris-HCl, pH 7.2, 1 mM Na₂EDTA) for 15 minutes. The DNA in the gel was transferred on Hybond N blotting membrane (Amersham), and then the DNA was immobilized on the blotting membrane with cross-linker (Spectrolinker XL-1500) under condition of UV 120 mJ/cm², and in a vacuum oven at 80° C. for 1 hour to thereby immobilize the DNA.

The blotting membrane was allowed to react on a pre-hybridization solution [6×SSPE (20×SSPE: 175.3 g/L NaCl, 31.2 g/L NaH₂PO₄.2H₂O, 7.4 g/L Na₂EDTA, pH 7.4), 0.5% SDS, 5× BFP (100× BFP: 2% BSA, 2% Ficoll-40,000, 2% PVP-360,000), 50 μg/mL denatured salmon sperm DNA, 10% dextrin sulfate] at 65° C. for at least 2 hours. To the reaction mixture, hybridization solution [6×SSPE, 0.5% SDS, 5× BFP, 250 μg/mL denatured salmon sperm DNA, 10% dextran sulfate] containing radioactive-labeled probe was added, and reacted at 65° C. for more than 16 hours. Thereafter, the reaction mixture was washed twice with Wash I solution (2×SSPE, 0.1% SDS) at room temperature for 15 minutes, and then twice with Wash II solution (1×SSPE, 0.1% SDS) at 65° C. for 15 minutes, to wash off non-specific hybridized probe. Finally, it was exposed on X-ray film (Kodak XAR film).

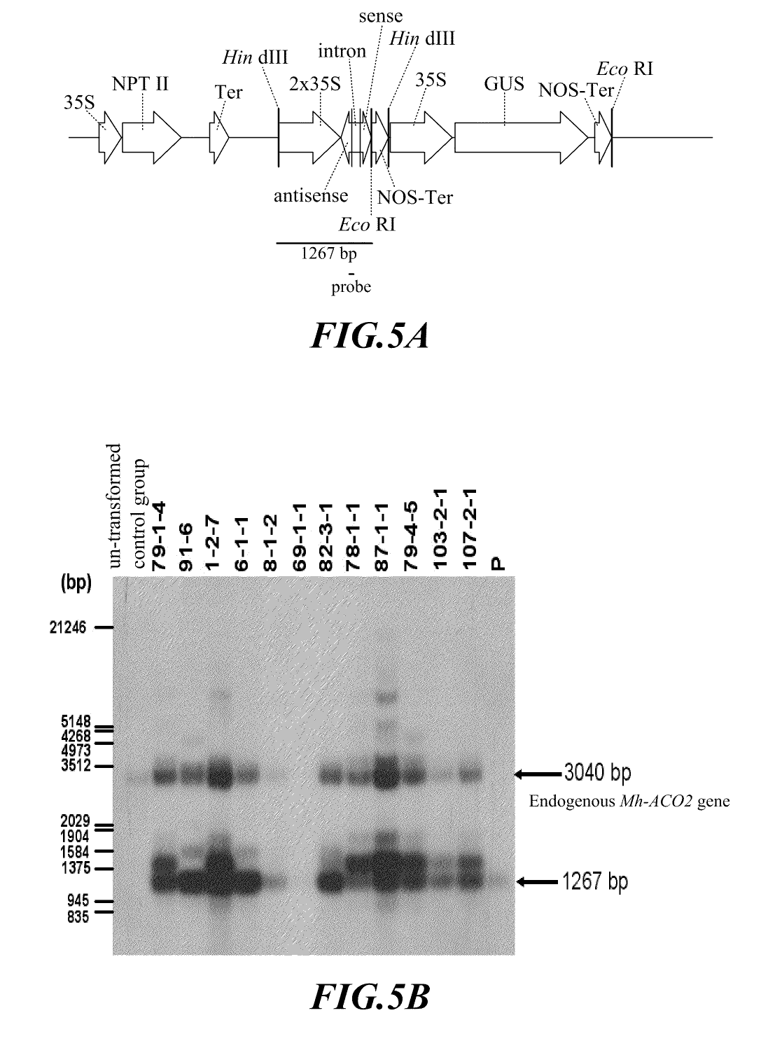

The test result indicated that as banana (Musa spp.) genome was digested at specific cleave site with EcoRI and HindIII, it was expected to obtain two DNA fragments of a size of 1,267 bp and 3,040 bp, respectively. Accordingly, different probes could be used to label exogenous gene. The result obtained from hybridization analysis by using Mh-ACO2 gene fragment as the probe (i.e. a plasmid pRNAi2ANS was double digested with restriction enzyme Xho I and SacII, the 160 bp Mh-ACO2 gene fragment thus-obtain was used as the probe, whose sequence was shown in SEQ ID No: 9) demonstrated that, a Mh-ACO2 gene fragment was present actually in the genome of transgenic plant. Though other than the expected fragment size, a signal of 3,040 bp fragment was also detected, there was no 3,040 bp size in the transformed DNA fragments. It was then suggested that this DNA fragment was an endogenous Mh-ACO2 gene fragment in the banana (Musa spp.) genome (FIG. 5A-B).

### EXAMPLE 4

### Observation of the Inhibition on the Transcription of Target Gene from RNA Level

1. The Observation of the Inhibition on the Transcription of the Transformed Gene with Reverse Transcription-Polymerase Chain Reaction (RT-PCR)

The total RNA extracted was used as a template, a reaction was carried out with One-Step RT-PCR Kit (GeneMark). The reaction mixture contained 0.1 μg/μL of template RNA, 50 ng/μL of primers, 1× Reaction Mix, 1× Enhancer, 2% Enzyme Mix. The reaction condition was at a temperature of 50° C. for 30 minutes, 94° C. 2 minutes, and then 35 cycles of 94° C. 30 seconds, 59° C. 30 seconds, and 72° C. 1 minute. Finally, it was reacted at 72° C. for 10 minutes, and then stored at 4° C. for later use. The primers used were shown as followed:

Forward primer IMAO-1: (containing the KpnI
restriction site)

(SEQ ID No: 3)

5′-ata [symbol] ccgcggaggtttgccatacttc-3′
KpnI
Reverse primer IMAO-2: (containing the BamHI
restriction site)

(SEQ ID No: 4)

5′-ata [symbol] gtcgacagctgcgagcagac-3′

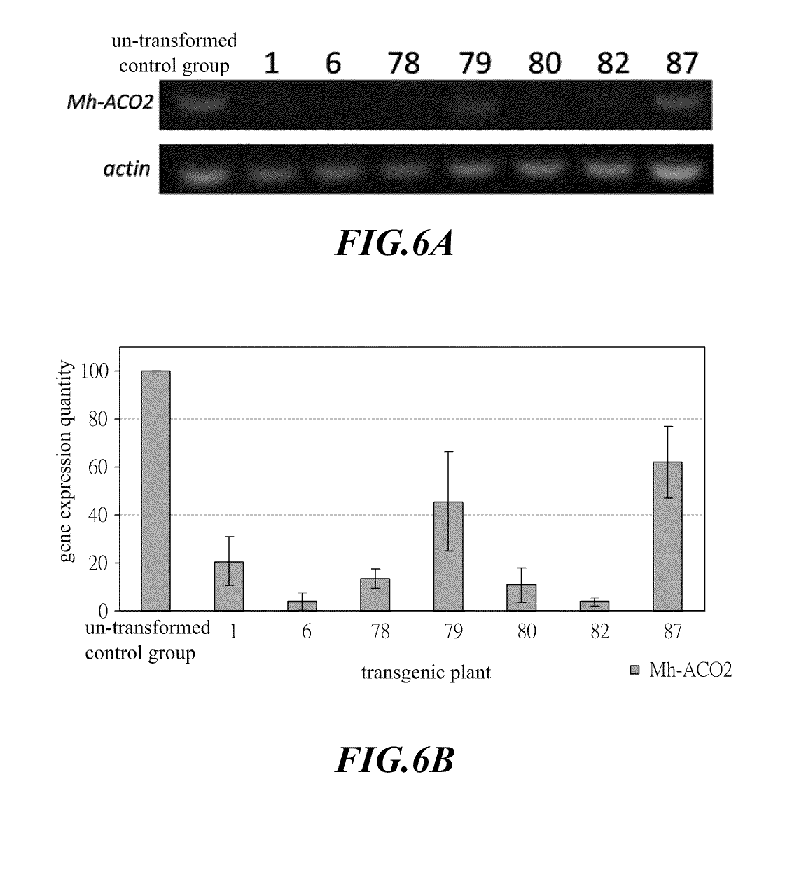

RT-PCR was used to detect the Mh-ACO2 expression among various transgenic plants, wherein the total RNA of new leaf tissue material was used. The result was shown in FIG. 6. Compared with the total RNA of new leaf tissue of an un-transformed Musa spp., Mh-ACO2 expression quantity in transformed plants was reduced. However, there was variation in the degree of the silencing effect among different transgenic plants. When the Mh-ACO2 expression quantity of the un-transformed control group was taken as 100%, Mh-ACO2 gene expression in the transgenic plants of the transgenic strain No. 2AS-1 was knocked-down 79.3%, that of the transgenic strain No. 2AS-6 was knocked-down 96.0%, that of the transgenic strain No. 2AS-78 was knocked-down 86.3%, that of the transgenic strain No. 2AS-79 was knocked-down 54.4%, that of the transgenic strain No. 2AS-80 was knocked-down 89.2%, that of the transgenic strain No. 2AS-82 was knocked-down 96.0%, and that of the transgenic strain No. 2AS-87 was knocked-down 37.8% (FIG. 6A-B).

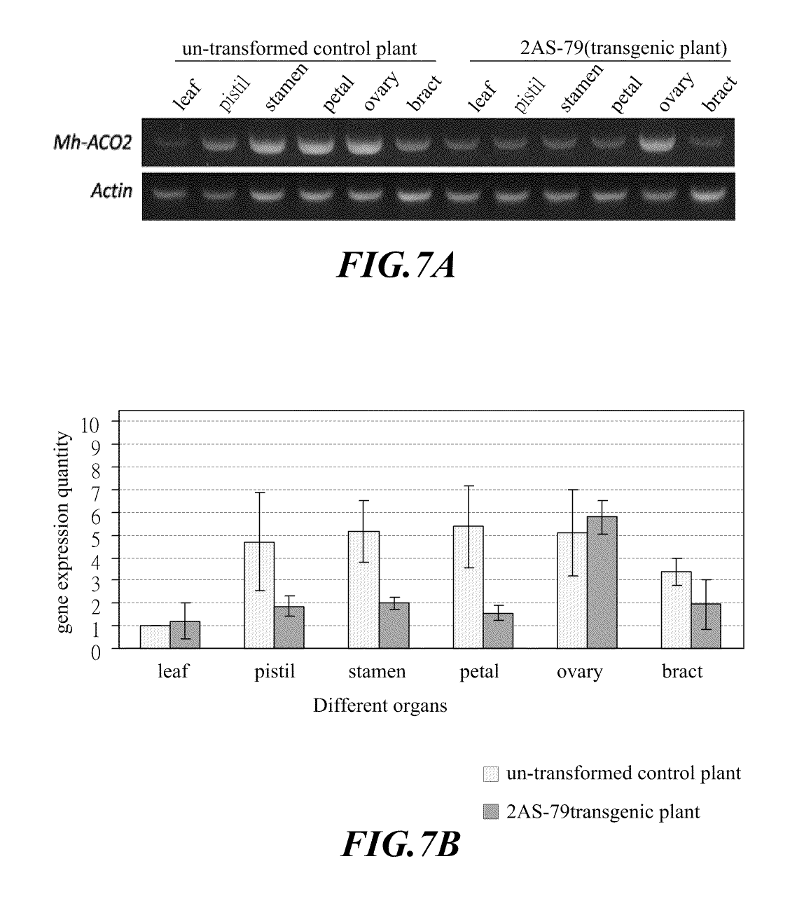

The Mh-ACO2 expression in tissues of leaf, stamen, pistil, petal, ovary and bract were observed between un-transformed control plant and Mh-ACO2-silenced transgenic plant. The result as shown in FIG. 7A-B indicated that, in un-transformed control group, except the less expression of Mh-ACO2 gene in leaf, it was found that Mh-ACO2 was mass expressed in reproductive organs of stamen, pistil, petal, ovary and bract. As compared with un-transformed control plant, the quantities of Mh-ACO2 gene expression in petal, stamen and pistil of transgenic plant indicated a significant silencing effect, i.e., 71.0% of Mh-ACO2 expression was inhibited in petal, the silencing effect in stamen was up to 61.5%, 60.5% of Mh-ACO2 expression quantity was knocked-down in pistil. Regarding the expression in leaf of transgenic plant, all transgenic plants had similarly low expression quantity. In addition, as compared with un-transformed control plant, expression of Mh-ACO2 had not been knocked-down in ovary and bract, with their expression quantities similar as those in plants of the control group (FIG. 7A-B).

### 2. The Observation on Transcription Inhibition of Transformed Gene by Small Fragment RNA Northern Hybridization Analysis

To the total RNA, 10 μL Urea loading dye (8 M urea, 20 mM EDTA-Na2, 5 mM Tris-HCl pH 7.5, 0.5% bromphenol blue) was added, the mixture was heated at 100° C. for 10 minutes, and then was stored on ice till used. Electrophoresis was carried out using 15% polyacryamide gel containing 8 M urea, and pre-heated 65° C. 1× TBE (10× TBE consisting of 0.9 M Tris, 0.9 M boric acid, 20 mM EDTA) as the electrophoresis solution, at voltage of 250 V. thereafter, RNA in the gel was blotted onto Hybond N nylon membrane (Amersham) with blotting electrophoresis chamber (Tanan VE-186) under conditions of using 0.5× TBE as the blotting electrophoresis buffer, voltage of 50 V, and blotting for one hour. The blotting membrane was then removed and air dried, cross-linked with UV 120 mJ/cm² as the cross-linker (Spectrolinker XL-1500), and then dried in vacuum at 80° C. for 1 hour to immobilize RNA. The preparation of nucleotide probe and the method for radioactive isotope labeling were carried out according to the Southern hybridization analysis described in Example 3.

The blotting membrane was allowed to react in a pre-hybridization solution (5×SSPE, 50% formamide, 0.5% SDS, 5× BFP) at 42° C. for at least 2 hours. Then, hybridization solution (5×SSPE, 0.5% SDS, 5× BFP, 200 μg/mL denatured salmon sperm DNA, 10% dextran sulfate) containing radioactive labeled probe was added and the mixture was allowed to react at room temperature for more than 16 hours. The reaction mixture was washed twice with Wash I solution (2×SSPE, 0.1% SDS) at room temperature for 15 minutes, then twice with Wash II solution (1×SSPE, 0.1% SDS) at 42° C. for 15 minutes, and finally, was exposed on X-ray film (Kodak XAR film) at −80° C.

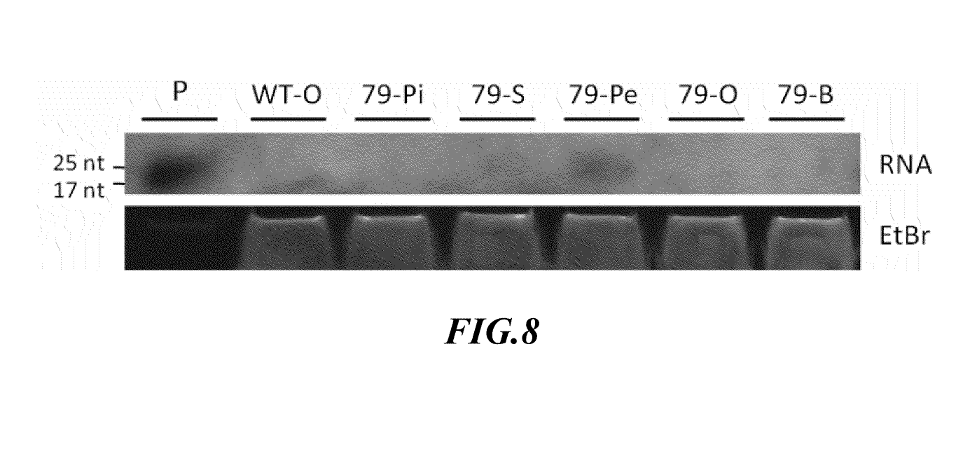

RNA interfering technique was used to silence target gene, thereby produced RNA fragments of 21 to 27 nt in size. Northern hybridization analysis was used to detect these small RNA fragments to confirm the interfering on expression by these RNA. Total RNA was extracted from the stamen, pistil, petal, ovary and bract tissues of transgenic plant No. 2AS-79. Thereafter, electrophoresis separation was carried out on RNA denatured polyacrylamide gel to separate RNA of a size less than 100 nt, and cDNA of Mh-ACO2 was used as probe to perform detection. The result as shown in FIG. 8 revealed that, in petal part of 2AS-79 transgenic plant, expression of siRNA could be detected, with fragment size of about 25-27 nt. This result indicated that, RNA interfering action was executed actually in the transgenic plant. Unfortunately, no significant siRNA expression was detected in stamen, pistil, ovary and bract tissues. This result indicated that the action of RNAi and the producing quantity of siRNA were varied among tissue organs (FIG. 8).

### EXAMPLE 5

### The Inhibition on the After-Ripening of Banana (Musa spp.) by Using Gene Transfer Technique

### 1. The Ripening Test on the Fruit of Banana (Musa spp.)

The fruit of banana (Musa spp.) was used in this test. The fingers in a green stage were rinsed separately. Notches were coated with vaseline, air dried and stored for later use. The natural ripening test consisted of placing various fingers at 25° C. to allow them ripening naturally. The general manner for estimating the ripening degree of the banana (Musa spp.) fruit comprised observation on the extent of color turning of pericarp, and then rating according to the fruit color index. Eight grades in total were classified between green pericarp color to the appearance of physiological flecks: the first grade was all green, the second grade was green—trace of yellow, the third grade was more green than yellow, the fourth grade was more yellow than green, the fifth grade was green tip, the sixth grade was all yellow, the seventh grade was yellow—flecked with brown, and the eighth grade was yellow with large brown areas.

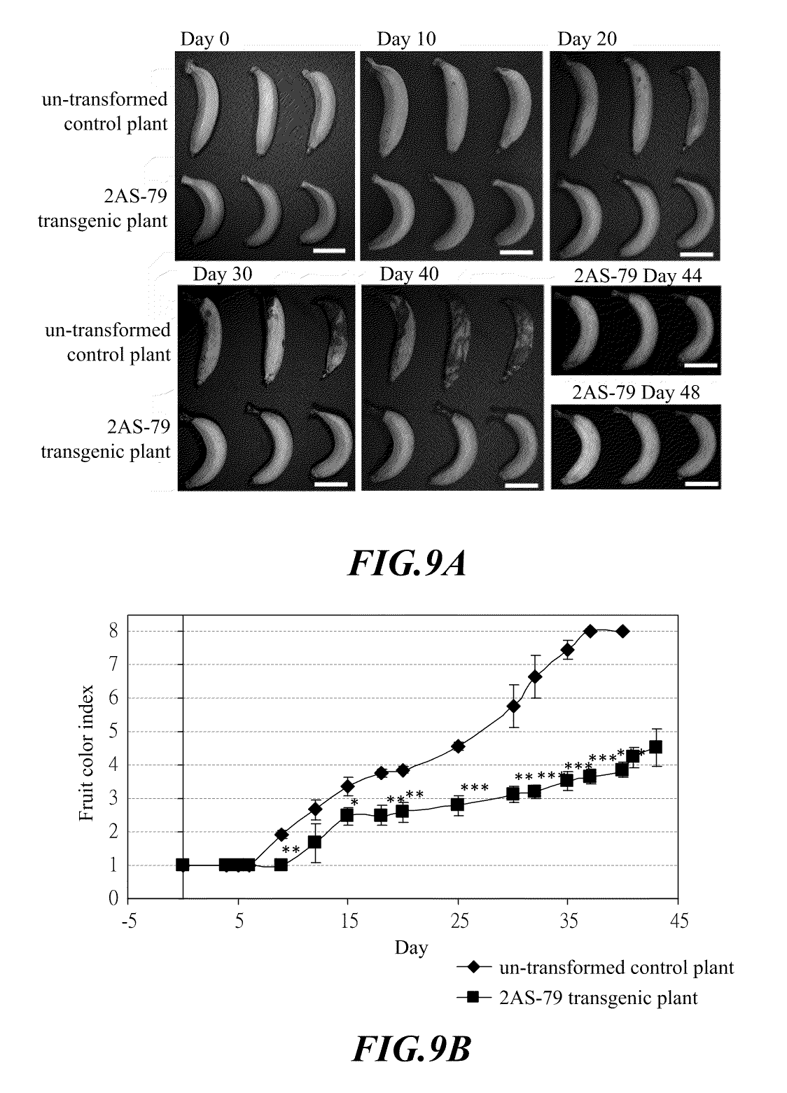

As shown in FIG. 9A-B, it could be found in natural ripening treatment that fruits of un-transformed control plants started slowly turning color after about Day 5, reached the third grade by about Day 15, the fourth grade after Day 20, the fifth grade at Day 28, the sixth grade at day 32, the seventh grade at Day 35, and the eighth grade at Day 37. Compared with the fruit of un-transformed control Musa spp., the fruit color turning of Mh-ACO2-silenced transgenic Musa spp. plant indicated significant delayed ripening, transgenic plant pericarp started to turn slowly its color after Day 6, reached third grade on about Day 20, and the time interval between the third grade and the fourth grade was maintained for approximately 20 days, reached the fourth grade on day 40, and reached the fifth grade on Day 43 (FIG. 9A-B).

### 2. The Determination of Respiration Rate in the Fruit of Banana (Musa spp.)

A single finger of the fruit of banana (Musa spp.) was weighed separately, and was placed in a tight-sealed 1-L respiration chamber, and was stood at 25° C. for 1 hour. 1 mL of gas in the respiration chamber was drawn and was subjected to a gas chromatograph [Shimadzu GC-8AIT, in combination with a thermal conductivity detector (TCD)] with separation column of ⅛″×6 ft stainless steel column packed with Porpark Q (80-100 mesh) to determine the amount of carbon dioxide, under conditions that the temperature in the oven containing the column was set at 40° C., the temperature on the injection port was set at 80° C.; and hydrogen gas was used as the carrier gas under a pressure set at 1 kg/cm². The height of the carbon dioxide peak obtained in the gas chromatography (GC) was used to calculate the respiration rate of the Musa spp. fruit: Respiration rate (ml

CO₂/g/hr)=[(Peak height of sample/Peak height blank)/Peak height of standard gas×concentration of standard gas (%)×1/100×total volume (ml)]/[Sample weight (g)×time (hr)]

The producing quantity of naturally ripened fruit determined by means of GC was used to calculate the respiration rate of the ripened fruit. The result shown in FIG. 10A revealed that, before Day 17, respiration rates of both the fruit of un-transformed control Musa spp. and transgenic Musa spp. were performed at low stationary quantity, and no significant difference was existed between these two groups, while after Day 18, the respiration rate of un-transformed control fruit started to increase at about 0.03 ml CO₂/g/hr, till it reached a respiration peak of 0.05 ml CO₂/g/hr on Day 27, and then began to decrease. In contrast, the fruit of transgenic plant maintained a low quantity and stable performance till the last test date of Day 32 (FIG. 10A).

### 3. The Determination of Producing Quantity of Ethylene in the Fruit of Banana (Musa spp.)

A single finger of banana (Musa spp.) fruit was weighed separately, was placed in tight-sealed 1-L respiration chamber, and stood at 25° C. for 1 hour. Then, 1 mL of the gas in the respiration chamber was drawn, and was analyzed on a gas chromatograph [CHROMPACK CP9001, in combination with a flame ionization detector (FID)], on a separation column of ⅛″×6 ft stainless steel column packed with active alumina (80-100 mesh) under conditions that the temperature in the oven containing the column was set at 90° C., the temperature at injection port was set at 150° C., the temperature of the detector was set at 130° C., hydrogen was used as the carrier gas under a pressure set at 20 kPa, and the burning gases was hydrogen and air. The height of ethylene peak obtained in the gas chromatography was used to calculate the producing quantity of ethylene of the Musa spp. fruit:

Producing quantity of ethylene (μl C₂H₄/g/hr)=[(Peak height of sample−Peak height of blank)/Peak height of standard gas×concentration of standard gas (ppm)×total volume (ml)]/[weight of sample (g)×time (hr)]

The quantity of ethylene produced by the naturally ripened fruit was determined by GC. The result shown in FIG. 10B indicated that, before Day 20, the producing quantities of ethylene by both the fruit of un-transformed Musa spp. fruit and the fruit of transgenic Musa spp. were performed at low stationary quantity, and no significant difference was existed between these two groups. After Day 20, the un-transformed control plant began mass production of ethylene, and a peak producing quantity of ethylene, about 1.7 μl C₂H₄/g/hr, occurred on about Day 27. Then, the production of ethylene was dropped abruptly. In contrast, the transgenic plant performed at an extremely low quantity, with only a small quantity of ethylene, about 0.2 μl C₂H₄g/hr, being detected on approximately Day 30 (FIG. 10B).

### EXAMPLE 6

### The Ripening Treated Transgenic Banana (Musa spp.)

### 1. The Ripening Test on the Fruit of Banana (Musa spp.)

Each finger of test banana (Musa spp.) fruit at the green ripe stage was rinsed separately. The notch thereof was coated with Vaseline. Then, they were dried naturally and stored for later use. The ripening test in this example adopted natural ripening and ripening by external application of ethylene, respectively. The natural ripening test comprised of placing each finger at 25° C. to allow them to ripen naturally. On the other hand, the ripening test by external application of ethylene comprised placing fruits in a respiration chamber containing ethylene at a concentration of 500 ppm and treated at 25° C. for 24 hours. Thereafter, the fruits of Musa spp. were removed from the respiration chamber, and the residual ethylene was removed in a hood, and then stored at 25° C. for allowing them to ripen.

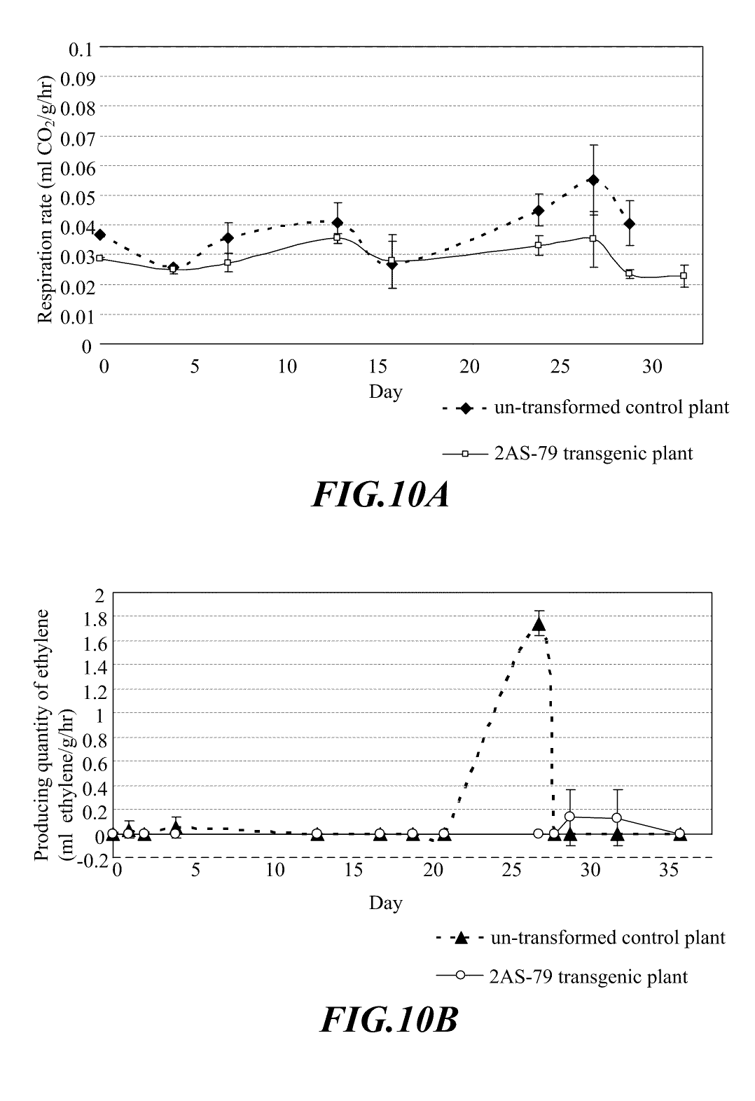

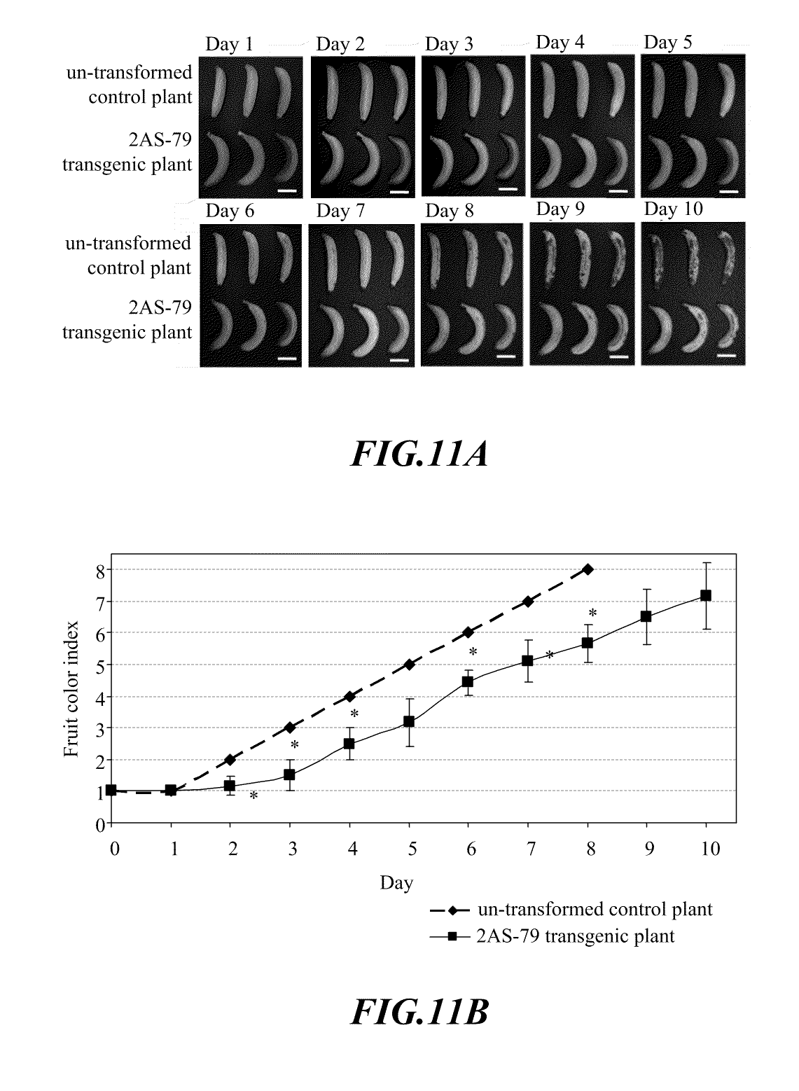

The day before ripening at Day 0, and the first day after ripening at Day 1 were taken as the basis. It was pointed out that, after ripening treatment, the grade of fruits of control group Musa spp. plant began to increase at a rate of one grade/day since Day 1 after ripening, and reached the eighth grade on Day 8. In contrast, fruits of transgenic plant began to turning color on Day 2, reached the second grade on Day 3, the third grade on the Day 4, and since then, color turning was slightly delayed that it reached just the fourth grade till Day 6. Thereafter, their grades were increased at a rate of one grade/day till reached the eighth grade on Day 10 (FIG. 11A-B).

2. The Determination Respiration Rate in the Fruit of Musa spp. after Ripening

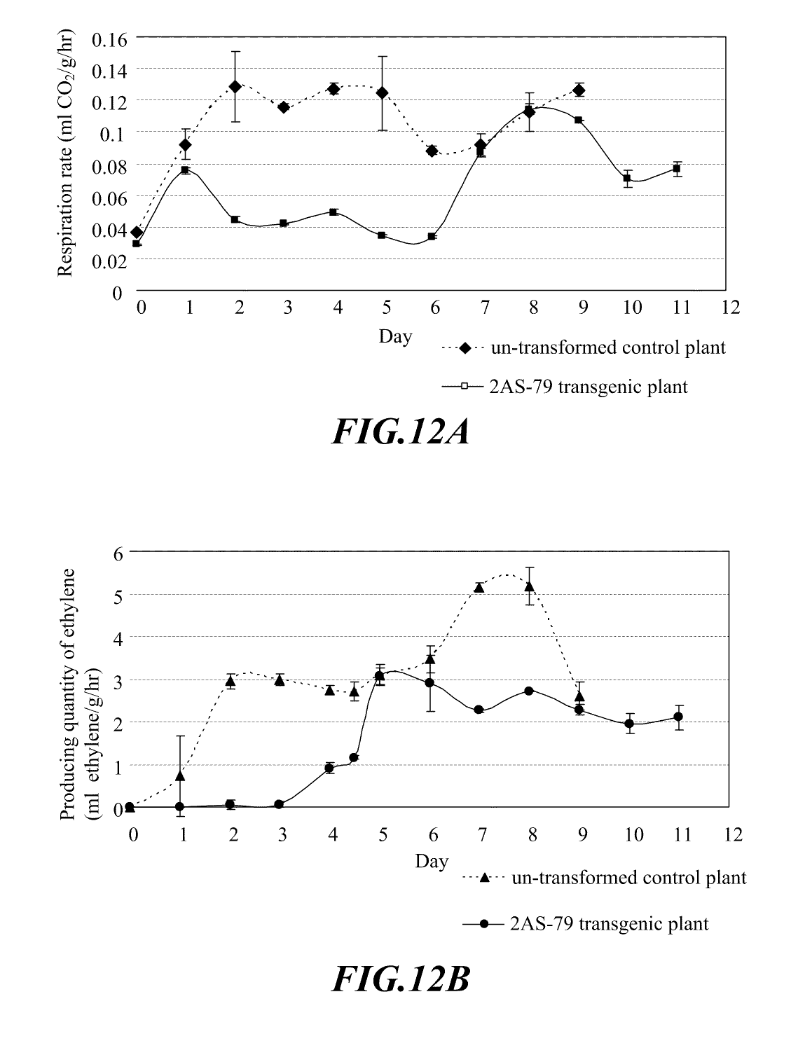

The quantity of carbon dioxide produced by the fruit after ripening was determined by GC so as to calculate the respiration rate of ripening fruits. The result indicated that, on Day 1 after ripening treatment, the respiration rate of fruits from un-transformed control plant increased immediately and reached their performance peak on Day 2, and maintained a stationary high respiration rate, about 0.13 ml CO₂/g/hr. The respiration rate began to decrease on Day 5, but increased again after Day 6. The respiration rate of transgenic plant performed similarly an abrupt increase on Day 1 of ripening treatment up to 0.075 ml CO₂/g/hr, while decreased subsequently on Day 2. Thereafter, a low respiration rate was maintained at about 0.04 ml CO₂/g/hr, and increased dramatically by Day 6. The respiration rate reached a peak value of 0.11 ml CO₂/g/hr on Day 8, after which, it began to decrease, and increased again after Day 10 (FIG. 12A).

### 3. The Determination of Producing Quantity of Ethylene in the Fruit of Banana (Musa spp.) After Ripening

The quantity of ethylene produced by the ripened fruit was determined by GC. The result indicated that, after Day 1 of ripening treatment, the quantities of ethylene produced by the fruit of un-transformed control group Musa spp. began to increase rapidly, and after reached a value of about 3 μl C₂H₄/g/hr on Day 2, maintained a stationary performance till Day 6. Then, the value began to increase again. After reaching a peak value of 5.2 μl C₂H₄g/hr on about Day 7, the producing quantity of ethylene decreased dramatically. On the other hand, the transgenic Musa spp. plant maintained a performance less than 0.1 μl C₂H₄/g/hr after ripening treatment, and increased only on Day 3. A peak value of 3.1 μl C₂H₄g/hr was reached on Day 5, and the producing quantity of ethylene decreased gradually and slowly but maintained at a value of between 2-3 μl C₂H₄g/hr (FIG. 12B). Therefore, it was possible to control the ripening time of said transgenic Musa spp. by artificial ripening treatment.

The composition and method for prolonging the shelf life of Musa spp. by using RNA interference provided according to the invention has following advantages over other conventional techniques:

1. The composition and method for prolonging the shelf life of banana (Musa spp.) by using interfering RNA provided according to the invention can control effectively the biosynthesis of ethylene in banana (Musa spp.), and can delay the ripening more effectively than ordinary banana (Musa spp.).

2. The composition and method for prolonging the shelf life of banana (Musa spp.) by using interfering RNA provided according to the invention, other than control effectively the biosynthesis of ethylene in banana (Musa spp.), can control the ripening time of banana (Musa spp.) by artificial ripening treatment, and thereby can greatly increase the economic value as well as the time frame of storage and transportation of banana (Musa spp.).

3. The gene transfer method for prolonging the shelf life of banana (Musa spp.) provided according to the invention can be applied for the gene transfer of banana (Musa spp.) more suitably and transfer efficiency than conventional gene transfer techniques.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A composition for prolonging the shelf life of banana by using interfering RNA, said composition comprising:
at least one interfering RNA, a gene transfer expression vector and a pharmaceutically acceptable carrier;
wherein said interfering RNA is linked to the 3′ end of the promoter in said gene transfer expression vector, and is constructed in said gene transfer expression vector in an order of following banana gene sequence: antisense strand—intron—sense strand; and wherein said interfering RNA prolongs the shelf life of banana by inhibiting the mRNA expression of enzymes associated with the biosynthesis of ethylene in banana.

2. A composition as in claim 1, wherein said interfering RNA has a sequence as shown in SEQ ID No:1, and is used to inhibit mRNA expression of Musa spp. ACC oxidase-1A and Musa spp. ACC oxidase-1B in banana, thereby inhibit/knock-down further the biosynthesis quantity of ethylene.

3. A composition as in claim 1, wherein said interfering RNA has a sequence as shown in SEQ ID No:2, and is used to inhibit mRNA expression of Musa spp. ACC oxidase-2 in Musa spp., thereby knock-down further the biosynthesis quantity of ethylene.

4. A composition as in claim 2, wherein the sequence of said interfering RNA compare with sequences of Musa spp. ACC oxidase-1A and Musa spp. ACC oxidase-1B, with at least 80% of sequence complementary, or at least 90% of sequence identity among them.

5. A composition as in claim 3, wherein the sequence of said interfering RNA compare with the sequence of Musa spp. ACC oxidase-2, with at least 80% of sequence complementary, or at least 90% of sequence identity between them.

6. A banana ACC oxidase control cassette, comprising:
a interfering RNA; and
a gene transfer expression vector;
wherein said interfering RNA is linked to the 3′ end of the promoter in said gene transfer expression vector, and is constructed in said gene transfer expression vector in an order of following banana gene sequence: antisense strand—intron—sense strand; and wherein said promoter activates the transcription of said interfering RNA in banana containing said banana ACC oxidase control cassette.

7. A control cassette as in claim 6, wherein said interfering RNA has a sequence as shown in SEQ ID No:1, and is used to inhibit mRNA expression of Musa spp. ACC oxidase-1A and Musa spp. ACC oxidase-1B in banana, thereby knock-down further the biosynthesis quantity of ethylene.

8. A control cassette as in claim 6, wherein said interfering RNA has a sequence as shown in SEQ ID No:2, and is used to inhibit mRNA expression of Musa spp. ACC oxidase-2 in Musa spp., thereby knock-down further the biosynthesis quantity of ethylene.

9. A control cassette as in claim 7, wherein the sequence of said interfering RNA compare with sequences of Musa spp. ACC oxidase-1A and Musa spp. ACC oxidase-1B, with at least 80% of sequence complementary, or at least 90% of sequence identity among them.

10. A control cassette as in claim 8, wherein the sequence of said interfering RNA compare with the sequence of Musa spp. ACC oxidase-2, with at least 80% of sequence complementary, or at least 90% of sequence identity between them.

11. A part of organ, tissue and cell of banana that has been subjected to gene transfer and hence contains a banana ACC oxidase control cassette as recited in claim 6.

12. A method for gene transferring in banana, said transferring method comprising following steps:
step 1 inducing callus:
cultivating male inflorescence of banana in an inducing medium to induce the formation of callus;
step 2 preparing cell suspension:
suspension culturing the callus obtained in step 1 in a suspension medium to obtain a homogeneous cell suspension;
step 3 co-culture transfer:
mixing suitable amount of callus or cell suspension and transformed Agrobacterium, co-culturing by shaking and then by standing at 25° C. for 2-4 days;
step 4 post-transfer screening:
after co-culturing by standing for 2-4 days, post-transferring said co-culture transformed cell in a solid post-transfer screening medium to carry out post-transfer screening;
step 5 somatic embryo differentiation:
after culturing in said solid post-transfer screening medium for two months, culturing the transformed cell in a regeneration medium till the formation of somatic embryo;
step 6 inducing multiple shoot:
after the formation of somatic embryo cells, culturing said somatic embryo cells in a multiple shoot-inducing medium to induce the germinating of multiple shoot and regenerating into plantlet;
step 7 inducing root:
as the plantlet growing to suitable size, shifting the plantlet in a root-inducing medium to induce rooting and promote the growth of the plant.

13. A method as in claim 12, wherein the co-culture transferring method described in said step 3 is one selected from the group consisting of Agrobacterium, genetic combination virus infection, transposon vector transfer, gene gun transfer, electroporation, microinjection, pollen tube pathway, liposome-mediated transfer, ultrasonic mediation transfer, silicon carbide fiber-mediated transformation, electrophoresis, laser microbeam, polyethylene glycol (PEG), calcium phosphate co-precipitation, and DEAE-dextran transformation.

14. A gene transformed method for banana, said transferring method comprising following steps:
step 1 inducing somatic embryo:
culturing fruit finger primodia or apical meristem in an inducing medium to induce the formation of somatic embryo cell;
step 2 cocultivation with Agrobacterium:
mixing somatic embryo cell obtained in step 1 and transformed Agrobacterium in an inducing medium to carry out co-culture transferring treatment;
step 3 post-transfer screening:
after cocultivation, shifting the transformed cell in an inducing medium containing suitable antibiotics to carry out the post-transfer screening;
step 4 cultivating to a plant:
shifting the screened transformed cell in an inducing medium containing suitable antibiotics, and cultivating for 6 to 8 months to obtain a transgenic plant.

15. A method as in claim 14, wherein the cocultivation method described in said step 2 is one selected from the group consisting of Agrobacterium, genetic recombination virus infection, transposon vector transfer, gene gun transfer, electroporation, microinjection, pollen tube pathway, liposome-mediated transfer, ultrasonic mediation transfer, silicon carbide fiber-mediated transformation, electrophoresis, laser microbeam, polyethylene glycol (PEG), calcium phosphate co-precipitation, and DEAE-dextran transformation.

16. A method for prolonging the shelf life of banana by using an interfering RNA, characterized in that said method comprising of transferring banana ACC oxidase control cassette into the callus induced from banana male inflorescence by means of the gene transfer method as described in claim 12, and culturing to generate the transgenic banana or part of organ, tissue or cell of said transgenic banana containing said banana ACC oxidase control cassette;
wherein said banana ACC oxidase control cassette, comprising:
a interfering RNA; and
a gene transfer expression vector;
wherein said interfering RNA is linked to the 3′ end of the promoter in said gene transfer expression vector, and is constructed in said gene transfer expression vector in an order of following banana gene sequence: antisense strand—intron—sense strand; and wherein said promoter activates the transcription of said interfering RNA in banana containing said banana ACC oxidase control cassette;
wherein said interfering RNA has a sequence as shown in SEQ ID No:1 or SEQ ID No:2.

17. A method for prolonging the shelf life of banana by using an interfering RNA, characterized in that said method comprising of transferring said banana ACC oxidase control cassette into somatic embryo cell induced from fruit finger primodia or apical meristem of banana by means of the gene transfer method as described in claim 14, and culturing to generate the transgenic banana or part of organ, tissue or cell of said transgenic banana containing said banana ACC oxidase control cassette;
wherein said banana ACC oxidase control cassette, comprising:
a interfering RNA; and
a gene transfer expression vector;
wherein said interfering RNA is linked to the 3′ end of the promoter in said gene transfer expression vector, and is constructed in said gene transfer expression vector in an order of following banana gene sequence: antisense strand—intron—sense strand; and wherein said promoter activates the transcription of said interfering RNA in banana containing said banana ACC oxidase control cassette;
wherein said interfering RNA has a sequence as shown in SEQ ID No:1 or SEQ ID No:2.


SEQUENCE LISTING (21 sequences)

SEQ ID NO: 1 - DNA, 388 bp, Musa
  Musa spp., cv. Hsien Jin Chiao, AAA group
gtctcctcgaagtccggagggttggacggccattcgttgtcgtctcgaagaacgaacacgtcctcccagtccacgttgtccaagcgttgaccatcgccttctttcaccagtgtgtccaacagctgaacgggtttggatccgcggaggtttgccatcttcaccccgctcctctcctctgctttcatggctatgctggtgtaaaggtactatgttcccgatgctctgtatcgtcgctcgcagctgtcgacggatccaaacccgttcagctgttggacacactggtgaaagaaggcgatggtcaacgcttggacaacgtggactgggaggacgtgttcgttcttcgagacgacaacgaatggccgtccaaccctccggacttcgaggagac

SEQ ID NO: 2 - DNA, 394 bp, Musa
  Musa spp., cv. Hsien Jin Chiao, AAA group
gccttcctatactggtcatcaagatcggggatctcagaaatgttggagacggggagatgacgcaggaaaaaggtgctttcccagtcgaggtggtcgatttctgagtcggcgttttccagtgctttgttggcgaactcggatccgcggaggtttgccatcttcaccccgctcctctcctctgctttcatggctatgctggtgtaaaggtactatgttcccgatgctctgtatcgtcgctcgcagctgtcgacggatccgagttcgccaacaaagcactggaaaacgccgactcagaaatcgaccacctcgactgggaaagcacctttttcctgcgtcatctccccgtctccaacatttctgagatccccgatcttgatgaccagtataggaaggc

SEQ ID NO: 3 - DNA, 31 bp, artificial sequence
  forward primer
ataggtaccccgcggaggtttgccatacttc

SEQ ID NO: 4 - DNA, 29 bp, artificial sequence
  reverse primer
ataggatccgtcgacagctgcgagcagac

SEQ ID NO: 5 - DNA, 25 bp, artificial sequence
  forward primer
tatggatccgagttcgccaacaaag

SEQ ID NO: 6 - DNA, 23 bp, artificial sequence
  reverse primer
ccagaattcgccttcctatactg

SEQ ID NO: 7 - DNA, 21 bp, artificial sequence
  forward primer
ataggatccaaacccgttcag

SEQ ID NO: 8 - DNA, 25 bp, artificial sequence
  reverse primer
catgaattcgtctcctcgaagtccg

SEQ ID NO: 9 - DNA, 160 bp, Musa
  Musa spp., cv. Hsien Jin Chiao, AAA group
tcgagaattaattcgccttcctatactggtcatcaagatcggggatctcagaaatgttggagacggggagatgacgcaggaaaaaggtgctttcccagtcgaggtggtcgatttctgagtcggcgttttccagtgctttgttggcgaactcggatccgcg

SEQ ID NO: 10 - DNA, 25 bp, artificial sequence
  forward primer
atggattcctttccggttatcgaca

SEQ ID NO: 11 - DNA, 20 bp, artificial sequence
  reverse primer
attccttcatcgccttccta

SEQ ID NO: 12 - DNA, 21 bp, artificial sequence
  forward primer
tagcggacgtaccacaggtat

SEQ ID NO: 13 - DNA, 21 bp, artificial sequence
  reverse primer
gtaagcaagcttctccttgat

SEQ ID NO: 14 - DNA, 1998 bp, Musa
  CDS (1)..(1998): Musa spp., cv. Hsien Jin Chiao, AAA group
tatagattattagctttaaggatataataatcattttattatactaatctcttacaacaaattaatttcaaaaactattcaagttaattaatatgataaacctcattaaaaaaaatcttttccattaggcgattaacaaaaatgacttaacacgagttaattaatgtgatagtcaagtttatgtatgtgcttaagctgacagtcgactgctatattatctaaaccgaactcaagataaaattaatttccttcacagaatttgactgccacgtttggcggctgctgctgtctgcgttgaccacgcattttttatgcgtctggattgtctcacgaagaacacacgaaatctactggggatacgtgtgtctgttcctggttgcttcctcggattgggacccctacaagatggcagagcgaagcttcgcccaccataaatagggcccataaccctctaattctcttcatccatccgagcggtaatacactcccaaagcttgcagcaatactcgctcctctctgtccattaatttcttagttgacatggcgattccggtcatcgatttctccaagttggatggcaaggaaagggccgaaaccatggcccggattgccaatggatgcgaggaatggggattctttcaggtttgccatacttcaccccgctcctctcctctgctttcatggctatgctggtgtaaaggtgctatgttcccgatgctctgtatcgtctgctcgcagctggtgaaccatgggattccggtcgagctgctggaacgcgtgaagaaggtcagctccgagtgctataagttgagggaggagcgcttcaagggatccaaacccgttcagctgttggacacactggtgaaagaaggcgatggtcaacgcttggacaacgtggactgggaggacgtgttcgttcttcaagacgacaacgaatggccgtccaaccctccggacttcgagtagagttcgcatgccgctgctgtgctcgagttttagttgctacgatagccacaaacccgatgacgatgtgatccgatgattgctctgcagggagaccatgaaggagtacagggaagaaatcaggaagctggcggagaaaatgatggaggtaatggacgagaatctgggcttcgaaaagggctgcatcaagaaagcattctctggggacggccagcacccgcccttcttcggcaccaaggtgagccactacccgccgtgcccgcgcctggacctggtgaagggccttcgcgcccacaccgacgccggcggcgtcatcctcctcttccaggacgaccaagtcggcggcctccagatgctcaaggacggccggtggatcgacgttcagcctttggccgacgccatcgtcataaacaccggagaccagatcgaggtcctcagcaacggtcgctacaagagcgcgtggcaccgggtgctcgccaccagccacggcaaccgccgctccatcgcttccttctacaacccctccctgaaggcgaccatcgctccagccgccggcgccgccaccgaggaagctgcccctcctgctctgtacccaaagtttctgttcggggactacatggacgtgtacgcgaagcagaagtacgagcccaaggaaccgagatttgaggcagtcagagctatttgaggatggagaagctgcgaatctattgttggattataggaggatataattcattgtactagtttggtgtctcatgcatggattacataattgcaaacatgatctgcatctgaataatggctgcttgttctcaggacacgcagtcgtcaatctgtaagtcggtgttcagaataaaaaccaagtgctatgtttcctcgtcacatcttctcgagttgaattcaaaggatagaaaaaagggaaaacatagttccccttttgcatcaagcatcttaccacgacagctagcaaccatagtcggcaaggcaacaacaatcttaaggaaaggctgtgacgaatgcttctggtaatacata

SEQ ID NO: 15 - DNA, 1865 bp, Musa
  CDS (1)..(1865): Musa spp., cv. Hsien Jin Chiao, AAA group
tgaagatggaggttttctgatgtccaaatgtgctcgtcgaatggttagtattttgttttcacaagagaaactaatatcattatatggtgttctttcgttgtcactcgcttaatttaattatatatctaccgtgaaacttgagaatataatttcccccatggctgttaccatcagataagcgtgactcactttactacgttaagtcttccgatatgccatcgtctcgtatggcatcggtgctggtggctgttcgtgagttaagagtcccacgtttggcggctgctgctgtctgcgttgaccacgcattttttatgcgtctggattgtctcacgaagaacacacgaaatctactggggatacgtgtgtctgttcctggttgcttcctcggattgggacccctacaagatggcagagcgaagcttcgcccaccataaatagggcccataaccctctaattctcttcatccatccgagcggtaatacactcccaaagcttgcagcaatactcgctcctctctgtccattaatttcttagttgacatggcgattccggtcatcgatttctccaagttggatggcaaggaaagggccgaaaccatggcccggattgccaatggatgcgaggaatggggattctttcaggtttgccatacttcaccccgctcctctcctctgctttcatggctatgctggtgtaaaggtgctatgttcccgatgctctgtatcgtctgctcgcagctggtgaaccatgggattccggtcgagctgctggaacgcgtgaagaaggtcagctccgagtgctataagttgagggaggagcgcttcgagggatccaaacccgttcagctgttggacacactggtgaaagaaggcgatggtcaacgcttggacaacgtggactgggaggacgtgttcgttcttcaagacgacaacgaatggccgtccaaccctccggacttcgagtagagttcgcatgccgctgctgtgctcgagttttagttgctacgatagccacaaacccgatgacgatgtgatccgatgattgctctgcagggagaccatgaaggagtacagggaagaaatcaggaagctggcggagaaaatgatggaggtaatggacgagaatctgggcttcgaaaagggctgcatcaagaaagcattctctggggacggccagcacccgcccttcttcggcaccaaggtgagccactacccgccgtgcccgcgcctggacctggtgaagggccttcgcgcccacaccgacgccggcggcgtcatcctcctcttccaggacgaccaagtcggcggcctccagatgctcaaggacggccggtggatcgacgttcagcctttggccgacgccatcgtcataaacaccggagaccagatcgaggtcctcagcaacggtcgctacaagagcgcgtggcaccgggtgctcgccaccagccacggcaaccgccgctccatcgcttccttctacaacccctccctgaaggcgaccatcgctccagccgccggcgccgccaccgaggaagctgcccctcctgctctgtacccaaagtttctgttcggggactacatggacgtgtacgcgaagcagaagtacgagcccaaggaaccgagatttgaggcagtcagagctatttgaggatggagaagctgcgaatctattgttggattataggaggatataattcattgtactagtttggtgtctcatgcatggattacataattgcaaacatgatctgcatctgaataatggctgcttgttctcaggacacgcagtcgtcaatctgtaagtcggtgttcagaataaaaaccaagtgctatgtttcctcgtcacatcttctcgagttgaattc

SEQ ID NO: 16 - DNA, 3718 bp, Musa
  CDS (1)..(3718): Musa spp., cv. Hsien Jin Chiao, AAA group
aagcttctttcacgctactgtagtgtctctgttgacaaaaggttctttggcattaggaagatgtagagcttgacttctagtttgggagtttgtgccctggtacaggaatcttctcactgtggtggtgaaccagtgagtgaagtcgatgttaaatgctaccgtcgacagtgtgatcgtgtgtctcaagtgggtttaacttgatgattcgagctggcttgctgcacttggttttcttacagtacgtgtgtgtctcacagacataaacatgactctgttgacaacagggtgaattggcatcatcatcctttacctgtccctgatttgagctgtagtgctgcgctcagccagcgttttatcgaagaagataaacatgcgtgagcctacatgcaccaaagcttgccggcaagtcatgcatagctgcaaacatgtgacaggcaccgaaccaacaattgaagaagatacgataaacatgcgtgagcctacatgcaccaaagcttgccgacaagtcatgtttgggtgcacaatgtgtcctcatcttacttgcatatctgctgttgcacaacagcagattgcatggaggtgtgttttccggcaatgcaatctttgatgttggttctcttttctctcttcttgcattgtttatagctctgtttcttgtgctcttcttttacgtagattcatagcgtagcttaagttgttatagattacctgttttactgggcaaacttgtgcaacccaggaatattcccatgtgcatcttcttcctgttttcctctgtcaaactgttctgttcatgatgaggcagcaccgaatctaagagaaatatcctaatgttgattgatttaacctcataaaacttgaagcagaatatgcttgccgctttcatgtgatcaattgaattgtttgcttgcttcacgagaacaccacattctgaacccattgctttcttgtggccaccaaccggagaagggagtctatataactagccgagcgaggattttcccatgacctgttcatctcacgtagagatggtgatttggttatagttatagcgatctatgatcgaagaatgagaaaatacccagataacggagatccatgcgtcaccagatggaacctcggccgagtgcggccgagtgacactgtttgcacaccggatacttcatgttcacggcaatggccgacatgccgaacagccatcgagcgttgaatgtaaggcaggaatggcccatttctcacatacgagagggatacgagtggaaagggcgctctaatgagctgtgaatcgaaacaatttctacctatcgatccctgttcttttgatatgaagtatagccaacaggtcaagagaagacgagtacacacgcatcgccgatgctgtgacgttactttctgaggttggcaatttgtcactacaatccaagcggaagccatgcacgcgagcgtcgccatggaagaactcaacaacatgatgccttcccgggtctcctcaaaggggagagaccgatggaagcagccaaacttggtccccgatcgtgatgggacgcgagaggtggaagcaaggagggtggagaaccaggccaaaggtggtggggctgagagatggccaactgggtcaccttatggaatcggctccgttacgtcttccactgctgttgctctcgtcgatagatccttctccaactttgcttcttcactcatttcgtccctcgacgtcaagaacgcctataaattgcctggtaatcagcagcacctagcacactccagatagaaagcacaagtgcaatcagggaagaaagagcgtgtcatggattcctttccggttatcgacatggagaagcttttgggaagggagagaggagcagccatggagatcctccgagatgcttgcgagaaatggggcttctttgaggtgctgaagcatacataactggttttgcttctttgaactatatatattgctaaaaatgtactatttgcgcatgcaatctgtgtgtagattttaaaccatggcatctcacattacctcatggatgaagtggagaaggtgaacaaagaacagtacaacaaatgcagggagcaaaagttcaacgagttcgccaacaaagcactggaaaacgccgactcagaaatcgatcacctcgactgggaaagcacctttttcctgcgtcatctccccgtctccaacatttctgagatccccgatcttgatgaccagtataggttgcacgatctgatcatgatgtcatcttctagccttgtcttttcaccttgctcatcgtttcgtttcttgggacgatgactgcgtgcaggaaggcgatgaaggaatttgctgcagcgatagagaagctggcagagcggctgctcgacttgctgggtgagaacctggagctggagaaggggtacctgaagaaagccttctctaatggatccaaggggccaacctttgggaccaaggtcagcagctacccgccatgcccgcgcccggacctggtgaagggcctgagggcgcacaccgacgccggaggcatcatcttgctcttccaggacgaccaggtcagcggcctgcagttcctcaaggacggcgagtggctggacgtgccccccatgcgccacgccatcgtcgtcaacctcggcgaccagctcgaggtttgggtcctctttgctctcgtttccgctgcccgtcgtctgtgatgttgaatgcaacgaggtctgcaggtaatcaccaatggcaagtacaagagcgtggtgcaccgcgtggtggctcagactgatggcaacaggatgtcgattgcctccttctacaaccccgggagcgacgctgtgatcttcccggcccccgctcttgtggagaaggaagcagaggagaagaaggaggtctatccgaggttcgtgttcgaggattacatgaagctctacgtcgggcataagttccaggccaaggagccaagattcgaagccatgaaagccatggaagcagttgccacccacccaatcgctacctcttaagtgacagcccccaagttagtgcatgtcgctgtacttcgcgttaggaagctgtcgtgtatgtctatgcaacccgatggaagcgtggtatgtacgtgtttgagccttttctaatgaagcaagtcatattatatatatatatatatatatatatatatataaataattactcttcaaaattttcttgaattttctcttttgttaatctttttatcccaatatttgataggaatccaaagattaagaaaaaagagtatggtaattaattagggatcatatatactgttttaatcaagaaaatttccagatttctggattagtggccaaccttaaggggattagtactaaaccgcccatctttacctatctaaacacagcctgccggaggagtcgaagaactacaaaaccctaaaccttgactactactcctcttccttccagaatcatggattcttctcctccctcgcatgccagatccgatcaagattcgctaccgaccgcctccgcagctccggaccagacgatcgcggacgggggaacaacggtggcggcggacgacggaggagaggagaagaagggagaagcgaatggaggaagcgaagtagaggaggagtgcggtttctgcctcttcatgaagggcggcgggtgcaaggatgcctttgtcgcgtgggagaaatgtatgcaggaagccgagaagcgcgacgaggacatc

SEQ ID NO: 17 - DNA, 104 bp, Musa
  Musa spp., cv. Hsien Jin Chiao, AAA group
aggtttgccatcttcaccccgctcctctcctctgctttcatggctatgctggtgtaaaggtactatgttcccgatgctctgtatcgtcgctcgcagctgtcgac

SEQ ID NO: 18 - DNA, 137 bp, Musa
  Musa spp., cv. Hsien Jin Chiao, AAA group
gccttcctatactggtcatcaagatcggggatctcagaaatgttggagacggggagatgacgcaggaaaaaggtgctttcccagtcgaggtggtcgatttctgagtcggcgttttccagtgctttgttggcgaactc

SEQ ID NO: 19 - DNA, 137 bp, Musa
  Musa spp., cv. Hsien Jin Chiao, AAA group
gagttcgccaacaaagcactggaaaacgccgactcagaaatcgaccacctcgactgggaaagcacctttttcctgcgtcatctccccgtctccaacatttctgagatccccgatcttgatgaccagtataggaaggc

SEQ ID NO: 20 - DNA, 134 bp, Musa
  Musa spp., cv. Hsien Jin Chiao, AAA group
gtctcctcgaagtccggagggttggacggccattcgttgtcgtctcgaagaacgaacacgtcctcccagtccacgttgtccaagcgttgaccatcgccttctttcaccagtgtgtccaacagctgaacgggttt

SEQ ID NO: 21 - DNA, 134 bp, Musa
  Musa spp., cv. Hsien Jin Chiao, AAA group
aaacccgttcagctgttggacacactggtgaaagaaggcgatggtcaacgcttggacaacgtggactgggaggacgtgttcgttcttcgagacgacaacgaatggccgtccaaccctccggacttcgaggagac